(19) United States
(12) Patent Application Publication
(10) Pub. No.: US 2026/0193314 A1
(43) Pub. Date: Jul. 9, 2026

(54) RATIOMETRIC FLUORESCENT SENSOR CONSTRUCTED BASED ON G PROTEIN-COUPLED RECEPTOR AND ITS CONSTRUCTION METHOD

(71) Applicant: Peking University, Beijing (CN)

(72) Inventors: Yulong Li, Beijing (CN); Yuqi Yan, Beijing (CN); Guochuan Li, Beijing (CN); Yipan Wang, Beijing (CN); Lei Wang, Beijing (CN); Jiesi Feng, Beijing (CN)

(21) Appl. No.: 19/433,833

(22) Filed: Dec. 28, 2025

(30) Foreign Application Priority Data
Jan. 6, 2025 (CN) 202510014341.2

(51) Int. Cl. C07K 14/705; G01N 33/94

(52) U.S. Cl. CPC C07K 14/70571; G01N 33/942; C07K 2319/60

(57) ABSTRACT

A ratiometric fluorescent sensor constructed based on a G protein-coupled receptor and its construction method are provided. It is discovered that by introducing a mutation at the same amino acid site (the third amino acid of the N-terminal linker) on a GRAB sensor, the GRAB sensor can be endowed with excitation ratiometric properties. The sensor with excitation ratiometric properties have high pH sensitivity, are not limited by the sensor's own expression level or external hardware conditions, maintain high stability of fluorescence brightness, can minimize fluorescence interference caused by motion, can sensitively detect neurotransmitter release, and have the potential for in vivo quantitative detection of neurotransmitter concentration.

### CROSS-REFERENCE TO RELATED APPLICATION

This application claims priority to Chinese Patent Application No. 202510014341.2, filed Jan. 6, 2025, which is herein incorporated by reference in its entirety.

### TECHNICAL FIELD

The disclosure relates to the field of biotechnologies, and more particularly to a ratiometric fluorescent sensor constructed based on a G protein-coupled receptor (GPCR).

### STATEMENT REGARDING SEQUENCE LISTING

The sequence listing associated with this application is provided in text format in lieu of a paper copy and is hereby incorporated by reference into the specification. The name of the XML file containing the sequence listing is 25097TBYX-USP1-SL.xml. The XML file is 27,254 bytes; is created on Dec. 20, 2025; and is being submitted electronically via patent center.

### BACKGROUND

Neurotransmitters, as the most important medium for signal transmission between neurons in the brain, have had their functions and modes of action extensively studied and reported. In addition, although there are many studies on the real-time dynamic changes of these neurotransmitters in different brain regions of the central nervous system, most existing tools lack high spatiotemporal resolution and cell specificity (Wu et al., “Pushing the frontiers: tools for monitoring neurotransmitters and neuromodulators”, Nat Rev Neurosci, 2022, Vol. 23, No. 5, pp. 257-274). For example, classical microdialysis, while capable of specifically detecting neurotransmitters and neuromodulators, has a low sampling frequency (once every 5 to 10 minutes), low spatial resolution, and lacks cell specificity (Chefer et al., “Overview of Brain Microdialysis”, Curr Protoc Neurosci, 2009, CHAPTER: Unit7.1, pp. 1-35). Fast-scan cyclic voltammetry has good temporal resolution but lacks cell specificity and selectivity (WERNER G. KUHR et al., “Real-time measurement of dopamine release in rat brain”, Brain Res, 1986, Vol. 381, No. 1, pp. 168-171; Robinson et al., “Detecting Subsecond Dopamine Release with Fast-Scan Cyclic Voltammetry in Vivo”, Clin Chem, 2003, Vol. 49, No. 10, pp. 1763-1773). In recent years, the research team led by Li Yulong, as one of the first internationally to develop GPCR Activation Based sensors (GRAB), has developed dozens of GRAB sensors for detecting different types of neurotransmitters, including dopamine, serotonin, norepinephrine, endocannabinoids, and neuropeptides. These novel genetically encoded fluorescent sensors effectively overcome the defects of existing detection methods, enabling real-time specific detection of neurotransmitters and modulators with high spatiotemporal resolution in specific cell types.

However, since current neurotransmitter sensors based on fluorescence intensity changes are influenced by many factors during use, such as chromophore number/expression level, fluorescence excitation intensity, and photobleaching, the application of such sensors based on single fluorescence intensity to reflect neurotransmitter changes is mainly limited to determining the occurrence of neurotransmitter release events caused by neuronal activity, and cannot be further used for sensitive and accurate measurement of neurotransmitter release concentration.

Therefore, the disclosure aims to develop a universal strategy capable of enhancing the excitation ratiometric properties of sensors, i.e., the property of exhibiting measurable fluorescence intensity changes at two distinct excitation wavelengths upon ligand binding. These sensors with excitation ratiometric properties can have high pH sensitivity, are not limited by the sensor's own expression level or external hardware conditions, maintain high stability of fluorescence brightness, can correct fluorescence interference caused by motion, can sensitively detect neurotransmitter release, and have the potential for in vivo quantitative detection of neurotransmitter concentration.

### SUMMARY

To provide improved fluorescent sensors with enhanced signal stability and quantitative capability, the purpose of the disclosure is to develop a universal strategy capable of enhancing the excitation ratiometric properties of GRAB sensors.

To achieve the above purpose, the disclosure adopts the following technical solutions.

The first aspect of the disclosure provides a ratiometric fluorescent sensor constructed based on a G protein-coupled receptor (GPCR).

In an embodiment, the ratiometric fluorescent sensor includes a GPCR, a circularly permuted fluorescent protein, and peptide-linkers. The peptide-linkers include an N-terminal linker and a C-terminal linker. The circularly permuted fluorescent protein is inserted between the fifth transmembrane domain and the sixth transmembrane domain of the GPCR, an N-terminus of the circularly permuted fluorescent protein is connected to the fifth transmembrane domain of the GPCR via the N-terminal linker. The C-terminus of the circularly permuted fluorescent protein is connected to the sixth transmembrane domain of the GPCR via the C-terminal linker.

In an embodiment, the N-terminal linker includes 5 amino acids, and the third amino acid of the N-terminal linker is mutated.

In an embodiment, the ratiometric fluorescent sensor can be expressed on the cell membrane; and the ratiometric fluorescent sensor can bind to a specific ligand when in contact with the specific ligand of the GPCR, thereby causing a detectable change in the fluorescence intensity of the fluorescent sensor.

In an embodiment, the GPCR is of human origin.

In an embodiment, the specific ligand includes a neurotransmitter.

In an embodiment, the neurotransmitter includes NTS, SST, PACAP, NPY, dopamine, and/or serotonin.

In an embodiment, the circularly permuted fluorescent protein is a circularly permuted enhanced green fluorescent protein (cpEGFP).

In an embodiment, when the cpEGFP is connected to the GPCR specifically binding the NTS to obtain a first resulting protein, the first resulting protein has the amino acid sequence as shown in SEQ ID NO: 1 or a variant thereof with a glutamic acid substitution at position 127.

In an embodiment, when the cpEGFP is connected to the GPCR specifically binding the SST to obtain a second resulting protein, the second resulting protein has the amino acid sequence as shown in SEQ ID NO: 2 or a variant thereof with a glutamic acid substitution at position 127.

In an embodiment, when the cpEGFP is connected to the GPCR specifically binding the PACAP to obtain a third resulting protein, the third resulting protein has the amino acid sequence as shown in SEQ ID NO: 3.

In an embodiment, when the cpEGFP is connected to the GPCR specifically binding the NPY to obtain a fourth resulting protein, the fourth resulting protein has the amino acid sequence as shown in SEQ ID NO: 4.

In an embodiment, when the cpEGFP is connected to the GPCR specifically binding the dopamine to obtain a fifth resulting protein, the fifth resulting protein has the amino acid sequence as shown in SEQ ID NO: 5 or a variant thereof with a valine substitution at position 127.

In an embodiment, when the cpEGFP is connected to the GPCR specifically binding the serotonin to obtain a sixth resulting protein, the sixth resulting protein has the amino acid sequence as shown in SEQ ID NO: 6.

In an embodiment, when the cpEGFP is connected to the GPCR specifically binding the NTS, the SST, the PACAP, the NPY, or the serotonin, the sequence of the N-terminal linker before mutation of the third amino acid is LEEGG (SEQ ID NO: 13).

In an embodiment, when the cpEGFP is connected to the GPCR specifically binding the NTS, the SST, or the NPY, the third amino acid of the N-terminal linker is mutated to leucine, and the mutated sequence of the N-terminal linker is LELGG (SEQ ID NO: 14).

In an embodiment, when the cpEGFP is connected to the GPCR specifically binding the PACAP, the third amino acid of the N-terminal linker is mutated to leucine, isoleucine, or methionine, and the mutated sequence of the N-terminal linker is any one of LELGG, LEIGG (SEQ ID NO: 15), or LEMGG (SEQ ID NO: 16).

In an embodiment, when the cpEGFP is connected to the GPCR specifically binding the serotonin, the third amino acid of the N-terminal linker is mutated to leucine or methionine, and the mutated sequence of the N-terminal linker is any one of LELGG or LEMGG.

In an embodiment, when the cpEGFP is connected to the GPCR specifically binding the dopamine, the sequence of the N-terminal linker before mutation of the third amino acid is LNSLL (SEQ ID NO: 17).

In an embodiment, when the cpEGFP is connected to the GPCR specifically binding acetylcholine, the sequence of the N-terminal linker before mutation of the third amino acid is ETEGG (SEQ ID NO: 21).

In an embodiment, when the cpEGFP is connected to the GPCR specifically binding the dopamine, the third amino acid of the N-terminal linker is mutated to leucine, alanine, or histidine, and the mutated sequence of the N-terminal linker is any one of LNLLL (SEQ ID NO: 18), LNALI (SEQ ID NO: 19), or LNHLI (SEQ ID NO: 20).

The applicant's team previously established a fluorescent sensor constructed based on GPCR, but such sensor based on single fluorescence intensity to reflect neurotransmitter changes are subject to many limitations. Therefore, the applicant sought mutation sites that could enhance the excitation ratiometric properties of GRAB sensors based on this foundation. The applicant discovered that by introducing variants at the same amino acid site (the third amino acid of the N-terminal linker), the excitation ratiometric performance could be enhanced in nearly ten types of GRAB sensors. These sensors with excitation ratiometric properties can have high pH sensitivity, are not limited by the sensor's own expression level or external hardware conditions, maintain high stability of fluorescence brightness, can correct fluorescence interference caused by motion, can sensitively detect neurotransmitter release, and have the potential for in vivo quantitative detection of neurotransmitter concentration.

The principle of the ratiometric fluorescent sensor constructed based on the GPCR (also referred to as the ratiometric GRAB fluorescent sensor in the disclosure) is as follows. The circularly permuted fluorescent protein is inserted between the fifth and sixth transmembrane domains of the GPCR; the binding of ligand to the GPCR induces a conformational change in the GPCR, which in turn causes a conformational change in the circularly permuted fluorescent protein, leading to a change in fluorescence signal intensity, thereby converting the ligand-induced GPCR conformational change into an optical signal change.

GPCRs are a large family of seven-transmembrane proteins expressed on the cell plasma membrane. The GPCR protein body consists of 7 transmembrane α-helical structures, with the N-terminus and 3 loops located extracellularly, and the C-terminus and 3 loops located intracellularly. The “G protein-coupled receptor (GPCR)” described herein is a large protein family of transmembrane receptors that sense extracellular molecules, activate intracellular signal transduction pathways, and ultimately trigger cellular responses. Ligands that bind and activate these receptors include light-sensitive compounds, odors, pheromones, hormones, and neurotransmitters, varying in size from small molecules to peptides to large proteins. GPCRs are involved in many diseases and are targets for about half of all modern medicinal drugs. Based on sequence homology and functional similarity, GPCRs can be classified into at least 5 classes: Class A rhodopsin-like, Class B secretin-like, Class C metabotropic/pheromone, Class D fungal pheromone, and Class E cyclic adenosine monophosphate (cAMP) receptors.

Class A rhodopsin-like receptors include: amine receptors; acetylcholine, α-adrenergic receptors, β-adrenergic receptors, dopamine, histamine, serotonin, octopamine, and trace amines; peptide receptors: angiotensin (NTS), bombesin, bradykinin, C5a anaphylatoxin, fMet-Leu-Phe, APJ-like, interleukin-8, chemokine receptors (C-C chemokine, C-X-C chemokine, BONZO receptor (CXCR6), C-X3-C chemokine and XC chemokine), CCK receptors, endothelin receptors, melanocortin receptors, neuropeptide Y (NPY) receptors, neurotensin receptors, opioid receptors, somatostatin (SST) receptors, tachykinin receptors (substance P (NK1), substance K (NK2), neurokinin B (NK3), tachykinin-like 1 and tachykinin-like 2), vasopressin-like receptors (vasopressin, oxytocin, and conopressin), galanin-like receptors (galanin, allatostatin, and GPR54), protease-activated-like receptors (e.g., thrombin), orexin & neuropeptide FF, urotensin II receptor, adrenomedullin (G10D) receptor, GPR37/endothelin B-like receptor, chemokine receptor-like receptor, and neuromedin U receptor; hormone protein receptors: follicle-stimulating hormone, luteinizing hormone-chorionic gonadotropin, thyroid-stimulating hormone, and gonadotropin; (Rhod) opsin receptors; olfactory receptors; prostanoid receptors: prostaglandins, prostacyclin, and thromboxane; nucleotide-like receptors: adenosine and purine receptors; cannabinoid receptors; platelet-activating factor receptor; gonadotropin-releasing hormone receptor; thyrotropin-releasing hormone & secretagogue receptors: thyrotropin-releasing hormone, growth hormone secretagogue, and growth hormone secretagogue; melatonin receptors; viral receptors; lysosphingolipid & lysophosphatidic acid abbreviated as LPA (endothelial differentiation gene abbreviated as EDG) receptors; leukotriene M receptors: leukotriene B4 receptor BLT1 and leukotriene B4 receptor BLT2; and Class A orphan/other receptors: platelet ADP & KI01 receptors, SREB, Mas oncogene, RDC1, ORPH, LGR-like (hormone receptor), GPR, GPR45-like, cysteinyl leukotriene, Mas-related receptors (MRGs), and GP40-like receptors.

Class B GPCRs (secretin receptor family) include polypeptide hormone receptors (calcitonin, corticotropin-releasing factor, gastric inhibitory peptide, glucagon, glucagon-like peptide-1, glucagon-like peptide-2, growth hormone-releasing hormone, parathyroid hormone, PACAP, secretin, vasoactive intestinal peptide, diuretic hormone, EMR1, latrophilin receptors), molecules thought to mediate cell-cell interactions at the plasma membrane (brain-specific angiogenesis inhibitor (BAI)), and a set of Drosophila proteins (Methuselah-like proteins) regulating stress response and lifespan.

Class C metabotropic glutamate/pheromone receptors include metabotropic glutamate, group I metabotropic glutamate, group II metabotropic glutamate, group III metabotropic glutamate, other metabotropic glutamate, extracellular calcium-sensing, putative pheromone receptors, GABA-B receptors (GABA-B receptor consists of two subunits (B1, B2), forming a dimeric protein), and orphan GPRC5 receptors.

GPCRs are involved in various physiological processes, including vision, olfaction, behavior and mood regulation, immune system activity and inflammation regulation, autonomic nervous system transmission, cell density sensing, and many others. It is known that inactive G proteins bind to the receptor in their inactive state. Once a ligand is recognized, the receptor or its subunit undergoes a conformational change, thereby mechanically activating the G protein, which dissociates from the receptor. The receptor can now activate another G protein or switch back to its inactive state. It is believed that receptor molecules exist in a conformational equilibrium between active and inactive biophysical states. Ligand binding to the receptor can shift the equilibrium toward the active receptor state.

GPCRs usable in the disclosure include but are not limited to acetylcholine receptors, dopamine (DA) receptors, serotonin (5-HT) receptors, neurotensin (NTS) receptor, neuropeptide Y (NPY) receptors, somatostatin (SST) receptors, Pituitary Adenylate Cyclase-Activating Polypeptide (PACAP) receptors. These receptors are known in the art to those skilled in the art, and their sequences can be obtained through various means, such as querying public databases.

Those skilled in the art can readily determine the N-terminus, transmembrane domains, intracellular loops, and C-terminus of a GPCR, for example, based on the similarity of its amino acid sequence to the transmembrane domains of known GPCRs. Various bioinformatics methods can be used to determine the location and structure of transmembrane domains in a protein, e.g., using BLAST or CLUSTALW programs for alignment and amino acid sequence comparison, which is routine in the related art. Based on alignment with known GPCRs containing transmembrane domains, those skilled in the art can predict the location and structure of transmembrane domains in other GPCRs. Many programs are also available for predicting the location and structure of transmembrane domains in proteins. For example, one or a combination of the following programs can be used: TMPred, which predicts transmembrane protein fragments; TopPred, which predicts membrane protein topology; PREDATOR, which predicts secondary structure from single and multiple sequences; TMAP, which predicts transmembrane regions in proteins from multiple aligned sequences; and ALOM2, which predicts transmembrane regions from a single sequence. According to standard nomenclature, transmembrane domains and intracellular loops are numbered relative to the N-terminus of the GPCR.

The term “circularly permuted fluorescent protein” as used herein is well-known to those skilled in the art and refers to a fluorescent protein formed by connecting the original molecular ends of the fluorescent protein and then cleaving the protein at any site to form new C-terminal and N-terminal ends. The fluorescent protein itself has its own chromophore center composed of 3 amino acids, and the chemical reaction it undergoes determines the spectral properties and fluorescence intensity of the fluorescent protein. Most of the chromophores of the fluorescent protein located inside the protein, protected by the surrounding β-folded barrel structure. When a fluorescent protein is fused to a target protein, pulling at the ends of the fluorescent protein can hardly cause changes in the environment around the chromophore, making it difficult to change the fluorescence intensity of the fluorescent protein. The chromophore of the circularly permuted fluorescent protein is relatively closer to the newly formed ends. When it is connected to a target protein, conformational changes in the target protein can pull the ends of the circularly permuted fluorescent protein, causing changes in the environment around the chromophore, thereby increasing or decreasing the fluorescence intensity of the fluorescent protein, thus converting the conformational change of the target protein into a change in its fluorescence intensity, enabling real-time detection via optical imaging methods. In specific embodiments of the disclosure, the circularly permuted fluorescent protein is cpEGFP. The cpEGFP can be the cpEGFP from GCAMP6s or GCAMP6m (Chen, T. W. et al., “Ultrasensitive fluorescent proteins for imaging neuronal activity”, Nature, 2013, Vol. 499, pp. 295-300), or the cpEGFP from GECO1.2 (Zhao, Y. et al., “An Expanded Palette of Genetically Encoded Ca²⁺ Indicators”, Science, 2011, Vol. 333, No. 6051, pp. 1888-1891). Their sequences can be obtained from the NCBI database or Addgene database.

The ratiometric fluorescent sensor constructed based on the GPCR (i.e., the ratiometric GRAB fluorescent sensor) of the disclosure should be able to be expressed on the cell membrane. Methods for detecting whether the sensor can be expressed on the cell membrane are well-known to those skilled in the art. For example, the sensor can be expressed in cells (e.g., HEK293T cells), and the expression morphology of the fluorescent protein in the cells can be analyzed. Proteins expressed on the cell membrane appear as a very thin ring at the outermost periphery of the cell. This can be compared with the brightfield channel to identify the cell outline for analysis. Sensors that fail to localize properly to the membrane often aggregate inside the cell, appearing as clustered signals within the cell under the microscope. Alternatively, it is also possible to quantify the measurement by co-expressing another known membrane-localized protein and calculating the colocalization between the fluorescent sensor signal and that protein.

The ratiometric fluorescent sensor constructed based on the GPCR of the disclosure should be able to bind to the specific ligand of the GPCR when in contact with it, thereby causing the fluorescence intensity of the sensor to have detectable changes at both 488 nanometers (nm) and 405 nm excitation, and further allowing calculation of specific ligand concentration changes via the Ex488/405 ratio. Methods for detecting this are known to those skilled in the art. For example, the sensor can be contacted with the specific ligand of the GPCR, and then fluorescence imaging of cells expressing the fluorescent sensor can be performed, taking continuous images before and after adding the ligand, and analyzing the fluorescence intensity changes before and after ligand addition to detect whether the fluorescent sensor has a fluorescent response to the specific ligand.

The terms “ligand” or “specific ligand” of the GPCR as used herein are used interchangeably and refer to molecules capable of binding and activating (or inhibiting) the GPCR, including light-sensitive compounds, odors, pheromones, hormones, and neurotransmitters. The binding between the GPCR and its ligand is highly specific, the ligand only binds to the specific receptor, and the receptor only binds to the specific ligand structure. The specificity of binding between the GPCR and its ligand means that the binding affinity of the GPCR for that ligand is significantly higher than its binding affinity for one or more other molecules. “Significantly” in “significantly higher” can refer to statistical significance. The ligands that different GPCRs can bind, or the GPCRs that different ligands can bind, are well-known to those skilled in the art.

The terms “peptide-linker” or “peptide-linker segment” as used herein are used interchangeably and refer to short peptides connecting the third intracellular loop of the GPCR and the circularly permuted fluorescent protein. In the disclosure, since the circularly permuted fluorescent protein is inserted into the third intracellular loop of the GPCR, the “peptide-linkers” described herein include the N-terminal linker located at the N-terminus of the circularly permuted fluorescent protein and the C-terminal linker located at the C-terminus of the circularly permuted fluorescent protein. In the disclosure, the function of the peptide-linkers is to assist in the correct folding of the fusion protein and to act as a bridge in transmitting receptor conformational changes to fluorescence protein brightness changes. Therefore, the peptide-linkers used should be those capable of performing the functions.

The second aspect of the disclosure provides a method for constructing a ratiometric GRAB fluorescent sensor.

In an embodiment, the method includes mutating the third amino acid of the N-terminal linker of the circularly permuted fluorescent protein connected to the GPCR.

In an embodiment, the circularly permuted fluorescent protein is cpEGFP, and the mutation includes mutating the third amino acid of the N-terminal linker of the cpEGFP to leucine.

In an embodiment, when the GPCR is the GPCR specifically binding the PACAP, the mutating further includes mutating the third amino acid of the N-terminal linker of the cpEGFP to isoleucine or methionine.

In an embodiment, when the GPCR is the GPCR specifically binding the dopamine, the mutating further includes mutating the third amino acid of the N-terminal linker of the cpEGFP to alanine or histidine.

In an embodiment, when the GPCR is the GPCR specifically binding the serotonin, the mutating further includes mutating the third amino acid of the N-terminal linker of the cpEGFP to methionine.

In an embodiment, the method further includes modifying the cpEGFP; the modification includes mutating a 127^{th} site of the cpEGFP.

In an embodiment, when the GPCR is the GPCR specifically binding the NTS or the SST, the mutation includes mutating the 127^{th} site of the cpEGFP to glutamic acid.

In an embodiment, the GPCR is a GPCR specifically binding the dopamine, the mutation includes mutating the 127^{th} site of the cpEGFP to valine.

The third aspect of the disclosure provides a biological material.

In an embodiment, the biological material includes:
1) a polynucleotide, wherein the polynucleotide encodes the ratiometric fluorescent sensor according to the first aspect of the disclosure;
2) an expression vector, wherein the expression vector includes the polynucleotide of 1);
3) a host cell, wherein the host cell includes the polynucleotide of 1) or the expression vector of 2).

The term “expression vector” refers to an expression vector capable of expressing a target protein in an appropriate host cell, which is a genetic construct containing operably linked basic regulatory elements. The operably linked basic regulatory elements allow expression of the inserted gene. Specifically, the recombinant vector is constructed to carry the encoding polynucleotide or a fragment thereof encoding the ratiometric GRAB fluorescent sensor of the disclosure, and the recombinant vector can be transformed or transfected into a host cell.

The expression vector of the disclosure can also be obtained by ligating (inserting) the polynucleotide of the disclosure into an appropriate vector. As long as it can replicate within a host, there is no particular limitation on the vector into which the gene of the disclosure will be inserted. For example, plasmid vectors, phage vectors, viral vectors, etc. can be used. Specifically, commercial expression vectors can be used, such as pDisplay vector, available from Invitrogen. Additionally, animal viruses such as retroviruses, adenoviruses, and vaccinia virus, and insect viruses such as baculovirus can be used. Plasmids usable in the disclosure are not limited to these examples.

The fourth aspect of the disclosure provides any one of the following applications, the application including:
1) an application of the method according to the third aspect of the disclosure in constructing the ratiometric GRAB fluorescent sensor;
2) an application of the ratiometric fluorescent sensor according to the third aspect of the disclosure in detecting concentration changes of a specific ligand of the GPCR in a sample to be tested or a tissue to be tested;
3) an application of the ratiometric GRAB sensor constructed by the method according to the third aspect of the disclosure in detecting concentration changes of a specific ligand of the GPCR in a sample to be tested or a tissue to be tested.

In an embodiment, the specific ligand is a neurotransmitter.

In an embodiment, the neurotransmitter includes NTS, SST, PACAP, NPY, dopamine, and/or serotonin.

In an embodiment, the detection includes qualitative and/or quantitative detection.

### BRIEF DESCRIPTION OF DRAWINGS

FIGS. 1A-1B show neuropeptide dynamic change detection and pH sensitivity of enhanced green fluorescent protein (EGFP). Specifically, FIG. 1A is a schematic diagram of neuropeptide sensor detecting neuropeptide release; and FIG. 1B shows the change of EGFP fluorescence brightness with pH.

FIGS. 2A-2B show chemical structure and spectrum of Fura-2 calcium dye. Specifically, FIG. 2A shows the chemical structure of Fura-2 calcium dye and the fluorescence ratio calculation method; and FIG. 2B shows the excitation spectrum of Fura-2 calcium dye.

FIGS. 3A-3B show spectra of wild-type green fluorescent protein (GFP) and EGFP fluorescent proteins. Specifically, FIG. 3A shows the spectrum of wild-type GFP, solid line is excitation spectrum, dashed line is emission spectrum; FIG. 3B shows the spectrum of EGFP, solid line is excitation spectrum, dashed line is emission spectrum.

FIG. 4 is a schematic diagram of an excitation spectrum model for a new generation ratiometric neuropeptide sensor; where the left part is a schematic diagram of non-ratiometric sensor excitation spectrum, the right part is excitation ratiometric neuropeptide sensor excitation spectrum; dashed lines represent the sensor without bound neuropeptide, solid lines represent the sensor with bound neuropeptide.

FIGS. 5A-5B are schematic diagrams of GFP spectrum and GFP undergoing excited state proton transfer (ESPT). Specifically, FIG. 5A shows the excitation spectrum (solid line) and emission spectrum (dashed line) of GFP; and FIG. 5B is a schematic diagram of GFP undergoing ESPT, the structures from left to right are Neutral form, State I, and Ionized form respectively.

FIG. 6 shows a schematic diagram of a structure-guided ratiometric sensor screening process.

FIGS. 7A-7B show structural prediction analysis of NTS1.3 sensor with cpEGFP. Specifically, FIG. 7A shows the Alphafold-predicted structure of the NTS1.3 sensor with cpEGFP; FIG. 7B shows the spatial distribution of 15 residues within 6 Å around the S205 residue, which can be grouped into four categories based on their spatial positions: residues in the linker region, residues adjacent to S205, residues near E222, and residues close to the chromophore; and FIG. 7C shows frequency of occurrence of each group of residues at increasing distances from the S205 residue.

FIGS. 8A-8B show screening results for the ratiometric NTS1.3 sensor with cpEGFP. Specifically, FIG. 8A shows spatial distribution of 15 residues within 6 Å around the S205 residue, which can be grouped into four categories according to their spatial positions: residues in the linker region, residues adjacent to S205, residues near E222, and residues close to the chromophore; and FIG. 8B shows saturation mutation screening results for the N3(E) site of the N-terminal linker of the NTS1.3 sensor with cpEGFP.

FIGS. 9A-9B show structural prediction and analysis NTS sensors. Specifically, FIG. 9A shows structural alignment of the NTS1.3 sensor with cpEGFP and the candidate NTS1.3-N3(E)L sensor with cpEGFP is presented, both predicted by AlphaFold; and FIG. 9B shows relative spatial positions of the N3(E), S205, and E222 residues in the NTS1.3 sensor with cpEGFP (top), and in the candidate NTS1.3-N3(E)L sensor with cpEGFP (bottom).

FIGS. 10A-10B show necessity of the main-chain carbonyl group at the S205 residue for ESPT in the NTS sensor. Specifically, FIG. 10A shows relative spatial positions of the N3(E)L, S205, and E222 residues in the NTS1.4 sensor with cpEGFP are shown (top), and those of the N3(E)L, S205A, and E222 residues (bottom); and FIG. 10B shows screening results of the S205 mutation in the NTS1.4 sensor, including ΔF/F₀ at 488 nm and 405 nm (left), and ΔR/R₀ and maximum brightness of the S205 mutant (right).

FIGS. 11A-11B show screening results of variants near the chromophore in the NTS1.3 sensor. Specifically, FIG. 11A is a schematic diagram of spatial distribution of amino acid residues near the chromophore in the GFP_S65T fluorescent protein; and FIG. 11B shows a summary of results for all mutant variants generated from the R168, Q94, and R96 residues in the NTS1.3 sensor.

FIGS. 12A-12B show screening results of variants related to folding kinetics in the NTS1.3 sensor. Specifically, FIG. 12A is a display diagram of amino acid residues that differ between cpEGFP and sfGFP in sequence; FIG. 12B shows a summary of repeated experimental results of the NTS1.3 mutants at the S30 and E39 residues.

FIGS. 13A-13D show development and optimization of ratiometric SST sensor. Specifically, FIG. 13A shows structural comparison between the SST1.0 sensor with cpEGFP and the candidate SST2.1 sensor with cpEGFP, both predicted by AlphaFold, showing the relative spatial positions of the N3(E), S205, and E222 residues in each sensor; FIG. 13B shows comparison of response amplitudes between SST2.1 and SST1.0 sensors; and FIG. 13C is a display diagram of amino acid residues that differ between cpEGFP and sfGFP in sequence (left), and FIG. 13D summarizes the repeated experimental results of mutant sensors after introducing variants at the S30 and E39 residues in the SST2.1 sensor.

FIGS. 14A-14D show optimization of ratiometric PACAP sensor. Specifically, FIG. 14A-14B shows structural comparison between the PACAP1.0 sensor with cpEGFP and the candidate PACAP1.0-N3(E)L sensor with cpEGFP, predicted by AlphaFold, showing relative spatial positions of the N3(E), S205, and E222 residues; FIG. 14B shows AlphaFold-predicted structure of the candidate PACAP1.0-N3(E)L sensor with cpEGFP, showing relative spatial positions of N3(E)L, S205, and E222 residues; FIG. 14C shows screening data for variants at the N3(E) position in the PACAP1.0 sensor; and FIG. 14D shows fluorescence response curves of three candidate sensors including PACAP-N3(E)I, PACAP-N3(E)L, and PACAP-N3(E)M, in response to varying concentrations of PACAP38, from left to right: response amplitude calculated under 488 nm excitation, response amplitude calculated under 405 nm excitation, and ratiometric response amplitude respectively.

FIGS. 15A-15B show structural prediction and analysis of NPY sensor. Specifically, FIG. 15A shows structural comparison between the NPY1.0 sensor with cpEGFP and the candidate NPY1.3 sensor with cpEGFP, both predicted by AlphaFold, showing relative spatial positions of the N3(E), S205, and E222 residues in each sensor (left: NPY1.0; right: NPY1.3); and FIG. 15B shows comparison of response amplitudes between NPY1.0 and NPY1.3 sensors.

FIGS. 16A-16B show structural prediction and analysis of GCG0.4 sensor. Specifically, FIG. 16A shows AlphaFold-predicted structure of the GCG0.4 sensor with cpEGFP, showing relative spatial positions of N3(L), S205, and E222 residues; and FIG. 16B shows GCG concentration-dependent fluorescence response curves of the GCG0.4 sensor, from left to right: response amplitudes calculated under 488 nm excitation, response amplitude calculated under 405 nm excitation, and ratiometric response amplitude respectively.

FIGS. 17A-17C show development of ratiometric DA sensors. Specifically, FIG. 17A shows excitation spectrum of dopamine sensor DA3m, dashed line indicates no DA present, solid line indicates 1 μM DA present; FIG. 17B shows screening results of variants at the N3(S) position in the DA3m sensor; and FIG. 17C shows screening results of variants at the S30 position in the DA3m sensor.

FIGS. 18A-18B show development of ratiometric 5-HT sensor. Specifically, FIG. 18A shows excitation spectrum of serotonin sensor 5-HT3.0, dashed line indicates no 5-HT present, solid line indicates 10 mM 5-HT present; and FIG. 18B shows screening results of variants at the N3(E) position in the 5-HT3.0 sensor.

FIGS. 19A-19C show development of ratiometric ACh sensor. Specifically, FIG. 19A shows excitation spectrum of acetylcholine sensor ACh41, dashed line indicates no ACh present, solid line indicates 1 mM ACh present; FIG. 19B shows an optimization process from ACh3.0 to ACh41 and comparison of their response amplitudes; FIG. 19C shows ACh concentration-dependent fluorescence response curves of the ACh41 sensor, from left to right: response amplitude calculated under 488 nm excitation, response amplitude calculated under 405 nm excitation, and ratiometric response amplitude respectively.

FIGS. 20A-20C show spectrum and pH sensitivity detection of NTS1.5 sensor. Specifically, FIG. 20A shows single-photon excitation (left) and emission (right) fluorescence spectra of the NTS1.5 sensor, dashed line indicates no NTS present, solid line indicates 1 M NTS present; FIG. 20B shows single-photon excitation spectra of the NTS1.5 sensor under different pH; FIG. 20C is a statistical plot of sensor fluorescence brightness changes with pH.

FIGS. 21A-21D show that ratio brightness of the NTS1.5 sensor is unaffected by expression level or laser power. Specifically, FIG. 21A is a bar graph showing Ex488/405, Ex488, and Ex405 signals of the NTS1.5 sensor at different expression levels; FIG. 21B is a statistical graph showing Ex488/405, Ex488, and Ex405 signals of the NTS1.5 sensor at different expression levels; FIG. 21C is a bar graph showing Ex488/405, Ex488, and Ex405 signals of the NTS1.5 sensor at different laser powers; FIG. 21D is a statistical graph of Ex488/405, Ex488, and Ex405 signals for the NTS1.5 sensor under different laser power levels.

FIGS. 22A-22C show spectrum and pH sensitivity detection of SST2.2 sensor. Specifically, FIG. 22A shows single-photon excitation (left) and emission (right) fluorescence spectra of the SST2.2 sensor, dashed line indicates no SST-14 present, solid line indicates 1 μM SST-14 present; FIG. 22B shows single-photon excitation spectra of the SST2.2 sensor under different pH; and FIG. 22C is a statistical graph of sensor fluorescence brightness changes with pH.

FIGS. 23A-23D show that the ratio brightness of the SST2.2 sensor is unaffected by expression level or laser power. Specifically, FIG. 23A is a bar graph of Ex488/405, Ex488, and Ex405 signals of the SST2.2 sensor at different expression levels; FIG. 23B is a statistical graph of Ex488/405, Ex488, and Ex405 signals of the SST2.2 sensor at different expression levels; FIG. 23C is a bar graph of Ex488/405, Ex488, and Ex405 signals of the SST2.2 sensor at different expression levels; FIG. 23D is a statistical graph of Ex488/405, Ex488, and Ex405 signals of the SST2.2 sensor at different expression levels.

### DETAILED DESCRIPTION OF EMBODIMENTS

To enable those in the related art to better understand technical solutions of the disclosure, the following further illustrates the technical solutions of the disclosure in conjunction with specific embodiments.

### Embodiment 1: Universal Development Strategy for Ratiometric Neuropeptide Fluorescent Sensors

Up to now, the applicant's team has successfully developed multiple neuropeptide sensors with higher response amplitudes and sensitivity. Taking NTS1.3 and SST2.0 sensors as examples, the performance of the NTS1.3 sensor is preliminarily validated at multiple levels including in vitro cultured cell level, neuron level, acute brain slice, and in vivo mouse brain level, proving its potential to monitor dynamic changes of NTS.

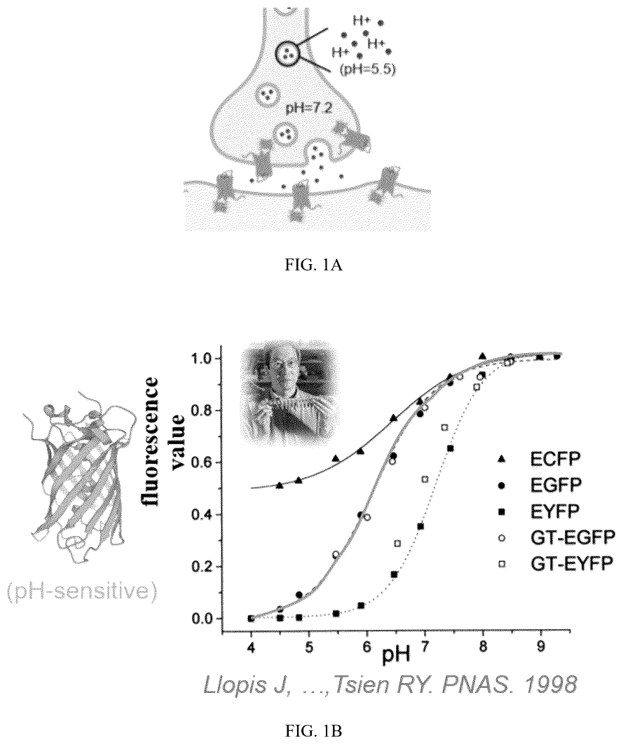

GRAB sensors couple conformational changes in GPCRs with circularly permuted EGFP (cpEGFP), using changes in cpEGFP fluorescence intensity to report dynamic ligand binding to GPCRs. In vivo, the cytoplasmic pH is 7-7.5, and the pH inside vesicles packaging neuropeptides is about 5.5. Upon fusion of these vesicles with the cell membrane and release of neuropeptides, the pH near the release site undergoes dramatic fluctuations (FIG. 1A). The cpEGFP is highly sensitive to environmental pH changes, and its fluorescence intensity decreases significantly with decreasing pH (FIG. 1), which greatly affects the signal specificity of the sensor when monitoring neuropeptide release in vivo.

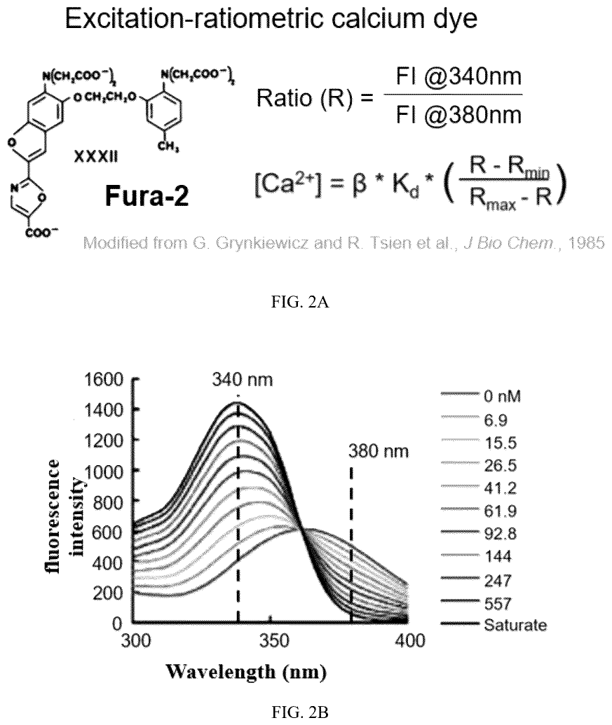

Roger Tsien developed the calcium dye Fura-2 as early as 1985 (FIG. 2A), whose most notable feature is its excitation ratiometric property, i.e., possessing two excitation peaks at Ex340 nm and Ex380 nm (FIG. 2B). By taking the ratio of fluorescence intensities excited at these two wavelengths under different calcium concentrations, a standard curve can be constructed for quantitative calcium measurements. However, a major drawback of Fura-2 is that both excitation wavelengths are in the blue region, prolonged irradiation of cells or tissues can cause photodamage and impair cellular or tissue viability.

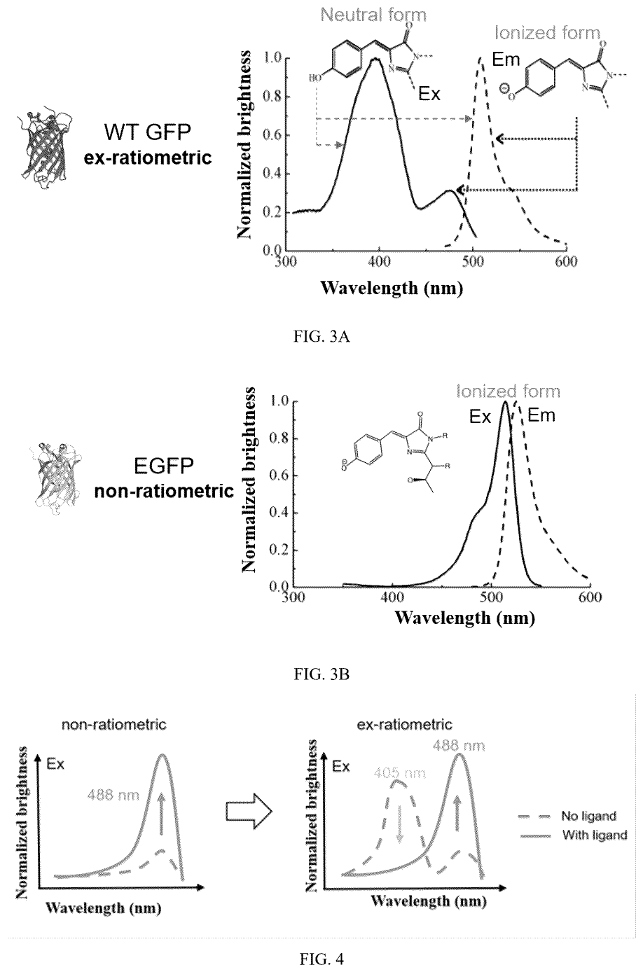

Based on the spectrum of wild-type GFP, it is found that wild-type GFP also has two excitation peaks, Ex405 nm and Ex488 nm (FIG. 3A), corresponding to the neutral form and ionized form of the fluorescent protein state, respectively. In this process, the neutral form undergoes excited-state proton transfer (ESPT), transitioning to an intermediate state (State I), which then emits fluorescence detectable by external instruments as changes in brightness. Unfortunately, EGFP, derived from wild-type GFP with three variants introduced into the chromophore and its surrounding region to enhance brightness, lost the Ex405 nm excitation peak and retains only the Ex488 nm excitation peak (FIG. 3B).

Based on this, this study proposes a hypothesis: whether it is possible to use AlphaFold for structural prediction and analysis of the cpEGFP-based sensor to identify key residues affecting the ESPT process, thereby restoring the ESPT network and promoting ESPT occurrence. Such an excitation ratiometric sensor would possess two excitation peaks, Ex405 nm and Ex488 nm, and upon binding to neuropeptides, it would elicit a negative response (off response) at 405 nm and a positive response (on response) at 488 nm (FIG. 4). Under these conditions, calculating the ratio of fluorescence signals at 488/405 nm (i.e., Ex488/405 ratio) would yield a larger response amplitude, and the ratio can also be used to correct signal fluctuations caused by pH, expression level, laser power, and other factors. Therefore, building upon the applicant's previous GRAB fluorescent sensors, this study aims to further endow the sensors with such excitation ratiometric properties, enhancing both response amplitude and signal stability.

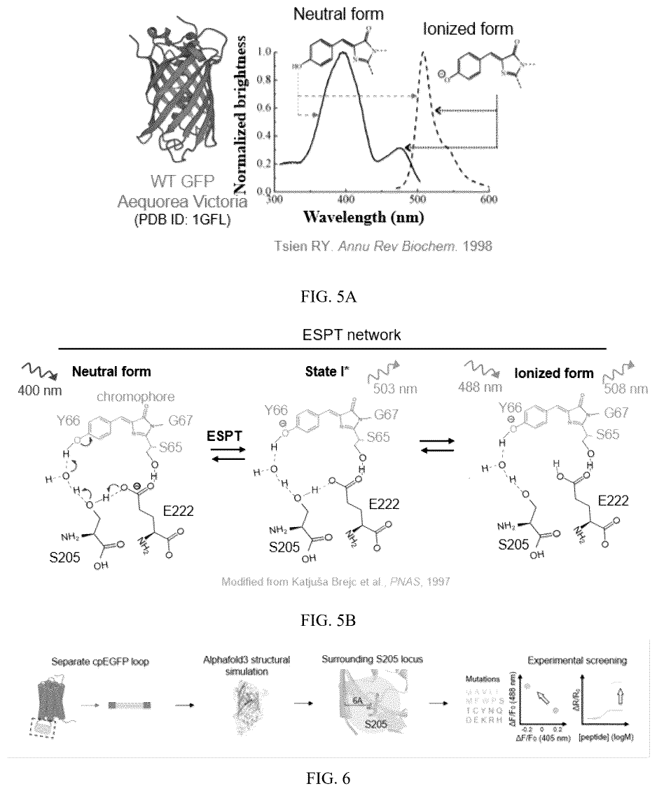

For candidate sensors that already exhibit higher response amplitudes, the next step is to rationally develop ratiometric neuropeptide sensors guided by structural insights. As introduced in the disclosure, for wild-type GFP, the fluorescent protein states under the two excitation peaks Ex405 and Ex488 correspond to the neutral form and ionized form, respectively (FIG. 5A). In this process, the neutral form undergoes ESPT, transitioning to an intermediate state (State I), which then emits fluorescence detectable as changes in brightness. In this network, proton transfer depends on the integrity of the ESPT network, composed of the chromophore, the main-chain carbonyl group at residue S205, the main-chain carboxyl group at residue E222, and water molecules (FIG. 5B). To endow GRAB sensors with excitation ratiometric properties, it is essential to enhance the response amplitude under 405 nm excitation, particularly increasing the fluorescence intensity at 405 nm in the unactivated state. Based on previous studies, mutating residues S205 and E222 in GFP to other amino acids has been shown to affect ESPT occurrence. For example, the E222Q mutation completely abolishes ESPT in GFP because the residue at position 222 loses its main-chain carboxyl group, thereby disrupting the ESPT network. In contrast, the S205V mutation reduces GFP's brightness but does not prevent ESPT from occurring. This suggests that the E222 residue is indispensable for ESPT. Therefore, directly altering the E222 site and its neighboring amino acid composition would likely inhibit ESPT occurrence and be unfavorable for developing ratiometric neuropeptide sensors.

Therefore, this study focuses on optimizing the amino acid composition near the S205 residue, aiming to identify candidate sensors with improved ratiometric properties and enhanced ESPT efficiency, resulting in a larger response amplitude under Ex405. First, the cpEGFP moiety and its flanking regions of five amino acids on each side are designated as the cpEGFP loop. Subsequently, AlphaFold3 is employed for structural prediction. Based on the predicted structure, amino acid residues within 6 Å of the S205 position are identified and subjected to saturation mutagenesis screening. The response amplitudes of the resulting mutants are then evaluated under Ex488, Ex405, and Ex488/405, abbreviated as ΔF/F₀ (488), ΔF/F₀ (405), and ΔR/R₀ (FIG. 6).

### Embodiment 2: Development of Ratiometric NTS Sensors

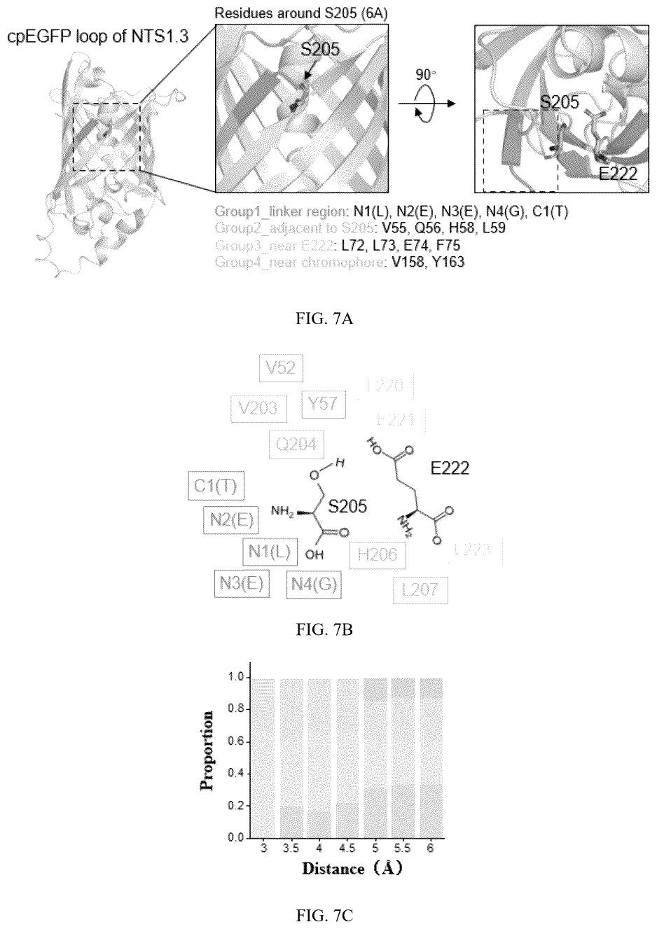

Taking the NTS1.3 sensor as an example, 15 amino acid sites are identified within 6 Å of the S205 residue (FIG. 7A). These amino acid sites are grouped into four categories based on their spatial positions: linker region, sites near S205, sites near E222, and sites near the chromophore (FIG. 7B). The sites near S205 and sites near E222 groups are located close to S205, with distances ranging from approximately 3.0 Å. Further expanding the screening distance range, it can be seen that amino acids in the linker group (N-linker) amino acids gradually appear (FIG. 7C), and all of these residues are positioned near the carbonyl and amide groups on the Cα of S205, specifically on the opposite side of the hydroxyl group of S205. Notably, the glutamic acid at the third amino acid of the N-linker, labeled N3(E), corresponds to position 146 in wild-type GFP. In the GFP ESPT network, position 146 is spatially adjacent to the ESPT network, suggesting its conformational changes are likely to affect ESPT occurrence.

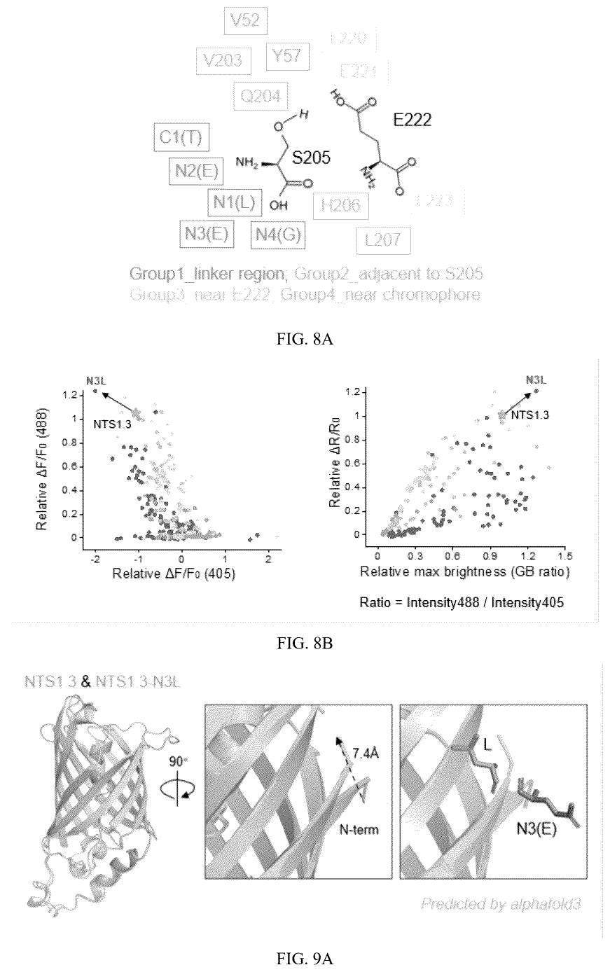

These 15 amino acids are spatially close to the S205 site (FIG. 8A) and are likely to promote sensor ESPT occurrence by altering their amino acid composition. Therefore, based on the NTS1.3 sensor, saturation mutagenesis screening is performed on these 15 sites, and the resulting mutants are excited at 488 nm and 405 nm to measure their response amplitudes after addition of 1 μM NTS. The results show that variants at amino acid residues near E222 and in the linker region significantly enhance ΔF/F₀(405) compared to the control sensor NTS1.3 (FIG. 8B). These mutants are further tested repeatedly on cells to ensure reliability of signal improvement. It is found that among the five mutation sites in the linker region, only when the glutamic acid at the N3(E) position is mutated to other amino acids could the candidate sensor exhibit improved brightness and response amplitude under both 488 nm and 405 nm excitation. Particularly, when the glutamic acid at N3(E) is mutated to leucine (L), the candidate sensor shows the greatest increase in brightness and response amplitude under both 488 nm and 405 nm excitation. In this study, the amino acid sequence of cpEGFP connected to the GPCR specifically binding NTS is shown as SEQ ID NO: 1: NYTKADKQKNGIKANFHTRINIEDGGVQLAYHYQQNTPIGDGPVLLPDNHYLSVQSK LSKDPNEKRDHMVLLEFVTAGTTLGMDELYKGGTGGSMVRKGEELFTGVVPILVEL DGDVNGHKFSVSGEGEGDATYGKLTLKFICTIGKLPVPWPTLVTTLTYGVQCFSRYP DHNKQHDFFKSAMPEGYVQERTIFFKDDGNYKTRAEVKFEGDTLVNRELEKGIDFKE DGNILGHKLEYN; its N-terminal linker is LEEGG, C-terminal linker is shown as SEQ ID NO: 22: TGATR; the sequence of the NTS1.3 sensor is shown as SEQ ID NO: 7: METDTLLLWULLWWPGSTGDTSLYKKVGTTGRLNSSAPGTPGTPAAQPFPQRAQAG LEBALLAPGEGNASGNASERVLAMPSSELDVNDTYSKVLVTAVYLALFVVGYVGNT VTAFTLARKKSLQSLQSTVHYHLGSLAISDLITLLLAMPVELVNFTWYHIPWAFGDA GCRGYYFLRDACTYATALNVASLSVERYLAICHPFKAKTLMSRSRTKRFISAIVLAAS VLLAVPMLFTMGQNRSGDQHASGLVCTPIMHTATVKVVIQVNTFMSFLFPMVVSVL NTTIANKLTVMVROAAEQQLNGAPGEPAPAGPRQTDALDLEEGGWYTKADKQKNG IKANFHTRINIEDGGVQLAYHYQQNTPIGDGPVLLPDNHYLSVQSKLSKDPNEKRDH MVLLEFVTAGTTLGMDELYKGGTGGSMVRKGEELFTGVVPILVELDGDVNGHKFSV SGEGEGDATYGKLTLKFICTIGKLPVPWPTLVTTLTYGVQCFSRYPDHNKQHDFFKSA MPEGYVQERTIFFKDDGNYKTRAEVKFEGDTLVNRELEKGIDFKEDGNILGHKLEYN TGATRRWRGREPNRVQALRHGVRVLRAVVIAFVVCWLPYHVRRLMFCYISDEQWT PFLYDFYHYFYMYTNALFYVSSTINPILYNLVSANFRHIFLATLACLCCPVWRRRKRP AFSRKADSVSSNHTLSSNATRETLY.

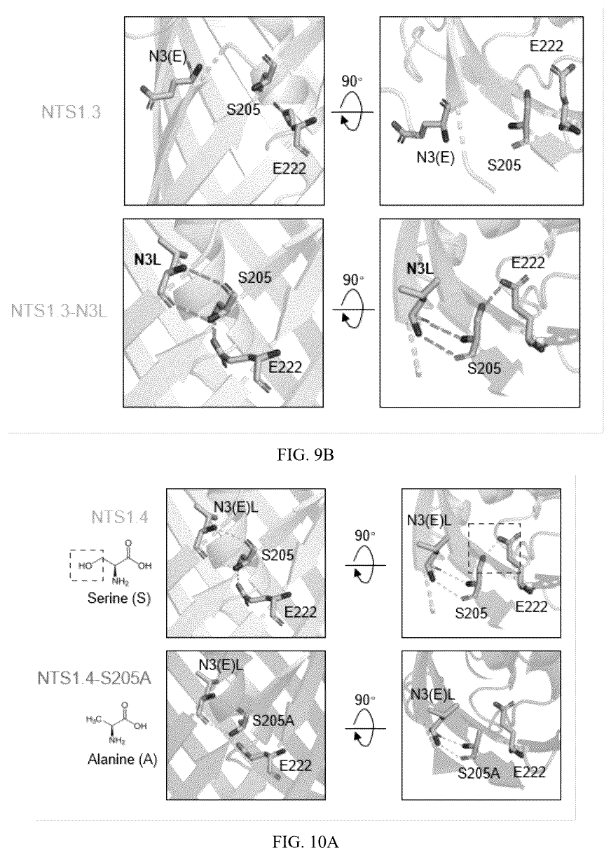

To analyze why mutating N3(E) to leucine (L) in the NTS1.3 sensor significantly enhances ESPT, its flanking regions (5 amino acids on each side) from both the NTS1.3 sensor and the candidate sensor NTS1.3-N3(E)L are extracted and subjected to structural prediction using AlphaFold. The predicted structures of the two sensors are highly similar; however, a notable conformational difference is observed in the N-terminal linker region of cpEGFP (FIG. 9A). Specifically, the N-terminal linker of cpEGFP in the candidate sensor NTS1.3-N3(E)L is positioned closer to the chromophore of cpEGFP and undergoes an inward shift of approximately 7.4 Å compared to that in the NTS1.3 sensor. This suggests that after the N3(E)L mutation, the N-terminal linker may form chemical bonds or interactions with certain residues on cpEGFP, leading to this significant structural rearrangement. Further analysis within 3 Å of the N3(E)L residue reveals that it can form two hydrogen bonds with the S205 residue, and such interactions are not observed in the NTS1.3 sensor (FIG. 9B). Additionally, in the predicted structure of NTS1.3-N3(E)L, a hydrogen bond between S205 and E222 can be observed, which is the structural basis for proton transfer in the ESPT network. The above results indicate that N3(E)L mutation stabilizes the orientation of the S205 residue through hydrogen bonding, thereby facilitating ESPT. Due to the significantly enhanced response amplitude under 405 nm excitation and a larger ratiometric response in the candidate sensor NTS1.3-N3(E)L, it is named NTS1.4.

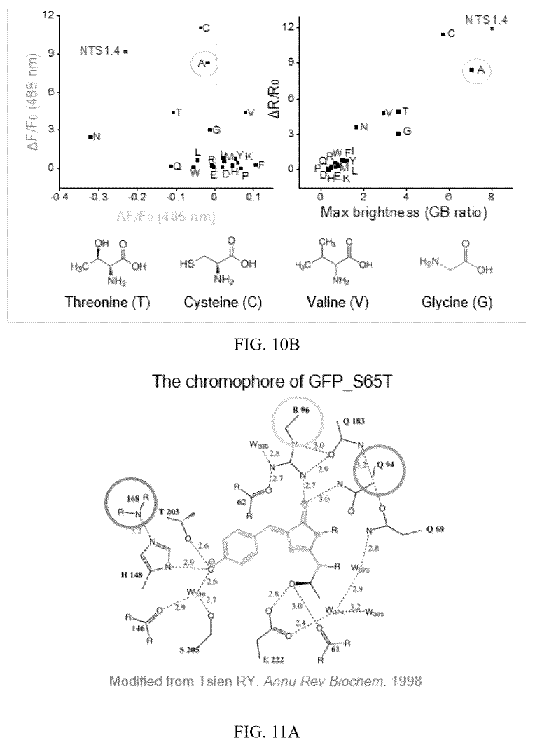

The S205 residue plays a critical bridging role in the ESPT network. It is hypothesized that the N3(E)L mutation promotes ESPT by forming hydrogen bonds with the side chain of S205. To further validate the necessity of S205 for ESPT in the sensor, mutagenesis experiments are designed to target the S205 position, particularly under conditions where the main-chain carbonyl group is absent, by assessing whether ESPT is affected, as reflected by changes in the response amplitude under 405 nm excitation. Taking mutation from serine (S) to alanine (A) as an example, structural prediction of the cpEGFP in both NTS1.4 and NTS1.4-S205A reveals that the absence of the main-chain carbonyl group at position 205 indeed disrupts the integrity of the ESPT network, likely suppressing ESPT (FIG. 10A). The response amplitude of the S205A mutant under 405 nm excitation is significantly reduced, while a slight decrease is observed under 488 nm excitation. In addition, compared to serine (S), other amino acids lacking the main-chain carbonyl group or substituted with different functional groups, such as threonine (T), cysteine (C), valine (V), and glycine (G), also exhibit significantly lower response amplitudes under 405 nm excitation than NTS1.4 (FIG. 10B). Therefore, S205 is necessary for ESPT occurrence in the sensor. Mutating Serine (S) to other amino acids lacking the main-chain carbonyl hydroxyl group blocks ESPT occurrence in the sensor, while N3(E)L mutation plays a supportive role within the ESPT network.

For neuropeptide sensors, leucine (L) at the N3 site indirectly promotes ESPT through hydrogen bonding with the side chain of S205. Whether other amino acid residues in the cpEGFP of the sensor can directly influence the ESPT network remains to be explored. To identify such residues, based on Roger Tsien's description of amino acid positions near the GFP chromophore (FIG. 11A), R168, T203, Q94, and R96 sites that might directly affect the chromophore are selected for mutation screening, using NTS1.3 and SST2.0 as templates, aiming to generate mutant sensors with enhanced response amplitudes under both Ex405 and Ex488/405 ratio. Results show that whether saturation mutagenesis screening at these four sites based on NTS1.3 sensor or based on SST2.0 sensor, although there are sites improving response amplitude under Ex405, the response amplitudes under Ex488 are significantly decreased in all these mutants, resulting in reduced ratio response (FIG. 11B).

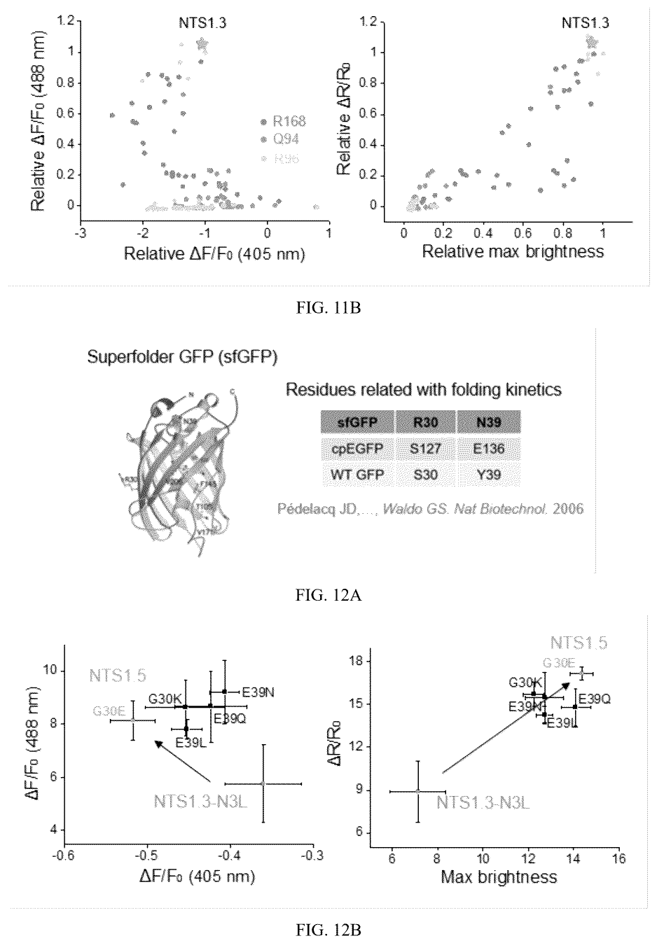

Besides residues near the chromophore that directly affect the ESPT network of cpEGFP, changes in the overall internal environment of the fluorescent protein (e.g., fluorescent protein folding) may also be likely to affect the ESPT network of cpEGFP. To further identify residues that could promote ESPT occurrence in the sensor, sequence alignment is performed between the cpEGFP used in the sensor and superfolder GFP (sfGFP), developed by Geoffrey S. Waldo's group, which is known for its high folding efficiency; residues differing between the two sequences are extracted (FIG. 12A). Specifically, position 30 corresponds to arginine (R) in sfGFP and serine (S) in cpEGFP, and position 39 corresponds to asparagine (N) in sfGFP and glutamic acid (E) in cpEGFP. The possible mechanisms by which variants at these two sites enhance fluorescent protein folding efficiency are described in the sfGFP publication. For example, when residue 30 is mutated from serine (S) is mutated to arginine (R), the R residue can form a complete network with adjacent E17 and E32 in the structure, potentially enhancing fluorescent protein folding rate.

Therefore, using the NTS1.4 sensor as a template, the amino acids at positions 30 (corresponding to position 30 in wild-type GFP and position 127 in cpEGFP) and 39 (corresponding to position 39 in wild-type GFP and position 136 in cpEGFP) are subjected to saturation mutagenesis screening. Results show that position 30 in cpEGFP (residue 127) is originally glycine (G); when it is mutated to E (glutamic acid), the mutant sensor shows significantly enhanced response amplitudes under both 488 nm and 405 nm excitation and in the ratio, and it is named NTS1.5 (FIG. 12B). Thus, based on the NTS1.3 sensor, by introducing the N3(E)L mutation in the N-terminal linker region and the G30E mutation in cpEGFP, the NTS1.5 sensor with enhanced excitation ratiometric properties is developed.

### Embodiment 3: Extending the Ratiometric Fluorescent Sensor Strategy to Other Neuropeptide Sensors

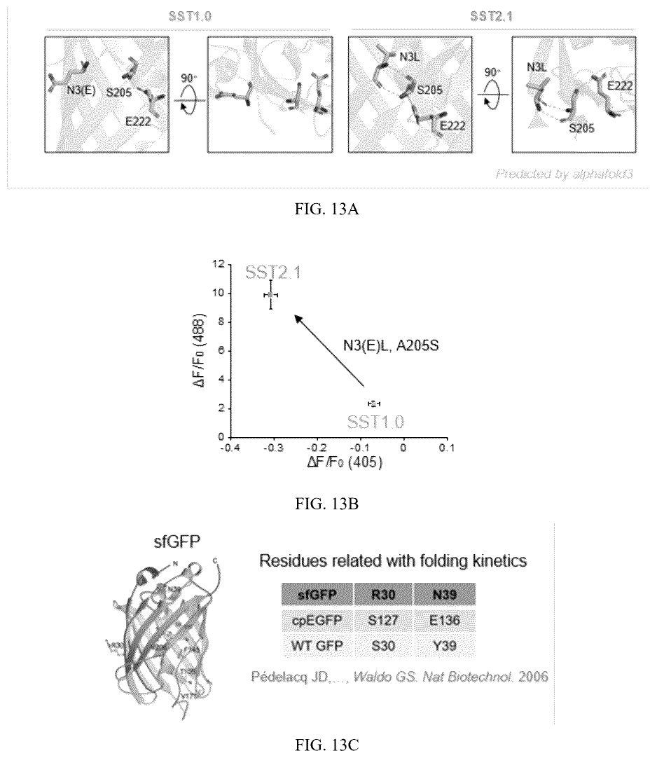

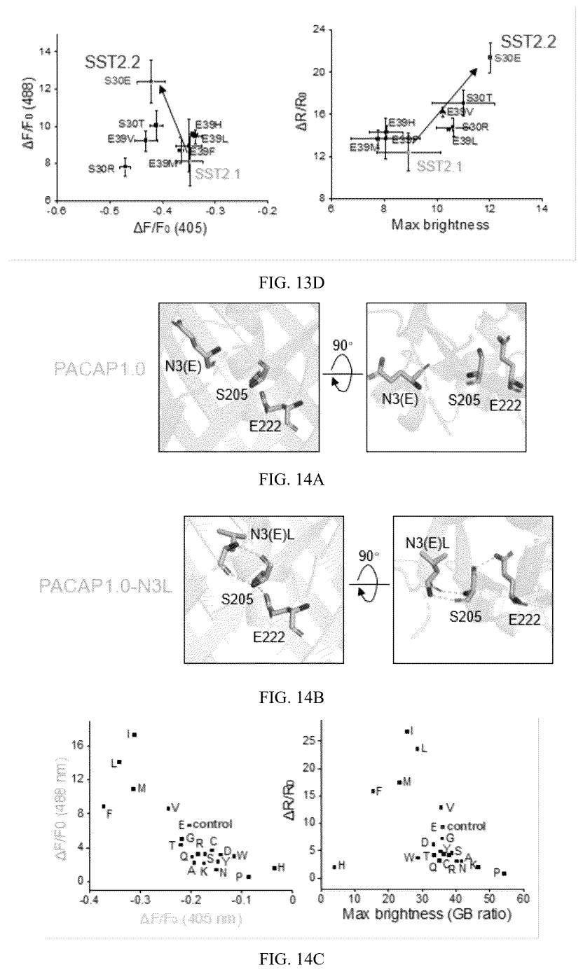

Similarly, in the optimization process of SST sensors, the N3(E)L mutation is also introduced. Predicted structures of cpEGFP in SST1.0 and SST2.1 sensors show that in cpEGFP of the SST2.1 sensor, N3(E)L can form two hydrogen bonds with the S205 residue, whereas such interactions are not observed in the SST1.0 sensor (FIG. 13A). Moreover, compared to SST1.0, SST2.1 exhibits significant improvements in both ΔF/F₀ (488) and ΔF/F₀ (405) (FIG. 13B). Using SST2.1 as a template, saturation mutagenesis screening is performed at positions 30 and 39 in cpEGFP (corresponding to residues 127 and 136 in cpEGFP), which are occupied by serine (S) and glutamic acid (E), respectively. Results show that when the serine (S) at position 30 is mutated to glutamic acid (E), the mutant sensor is demonstrated significantly enhanced response amplitudes under Ex488, Ex405, and Ex488/405 ratio are significantly improved, and it is named SST2.2 (FIG. 13C). In this study, the amino acid sequence of cpEGFP connected to the GPCR specifically binding SST is NVYIKADKQKNGIKANFHIRINIEDGGVQLAYHYQQNTPIGDGPVLLPDNHYLSVQS KLSKDPNEKRDHMVLLEFVTAGTTLGMDELYKGGTGGSMVRKGEELFTGVVPILVE LDGDVNGHKFSVSGEGEGDATYGKLTLKFICTIGKLPVPWPTLVTTLTYGVQCFSRY PDHNKQHDFFKSAMPEGYVQERTIFFKDDGNYKTRAEVKFEGDTLVNRELEKGIDFK EDGNILGHKLEYN (SEQ ID NO: 2); its N-terminal linker is LEEGG, C-terminal linker is TGAAA (SEQ ID NO: 23); the sequence of SST2.1 after N3(E)L mutation is METDTLLLWULLWWPGSTGDTSLYKKVGTTGEPLPPASTPSWNASSPGAASGGGDR TILVGPAPSAGAAVLVPVLVILLVCAGLGGNTLYVVILRFAMMTVTVNITILLALAVA DVLVMLGLPFLATQNAASPWPFGPVLCRLVMTLDGVNQFTSVFCLTVMSVDRYLAV VHPLSSARWRPRVAKLASAAAWLSLCMSLPLLVFAAQYQEGTCNASWPEPVGLWG AVFIIYTAVLGFPAPLLVICLCVLLTVVVRAGVWYGCALDLELGGNYIKADKQKNGI KANFHIRINIEDGGVQLAYHYQQNTPIGDGPVLLPDNHYLSVQSKLSKDPNEKRDHM VLLEFVTAGTTLGMDELYKGGTGGSMVRKGEELFTGVVPILVELDGDVNGHKFSVS GEGEGDATYGKLTLKFICTIGKLPVPWPTLVTTLTYGVQCFSRYPDHNKQHDFFKSA MPEGYVQERTIFFKDDGNYKTRAEVKFEGDTLVNRELEKGIDFKEDGNILGHKLEYN TGAAKRWRGQLKVWNYLVVILVFACCPLPPFTVNIVVILAVALPQEPASAGLYFFVVI LSYANSCANPVLVGFLSPNFRQSFQKVLCLRKGSGARDADATEPRPDRIRQQQEATPP AHRAAMCLMQTSKL (SEQ ID NO: 8).

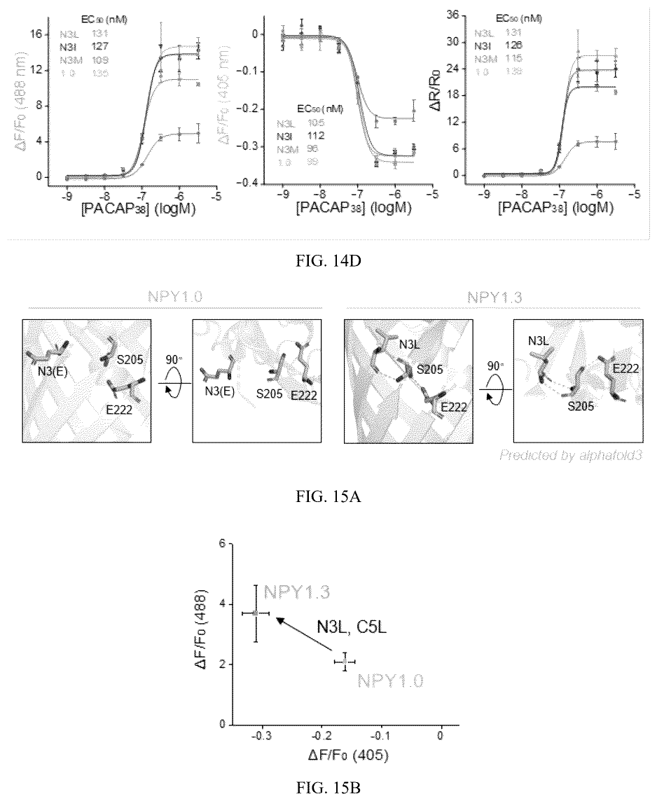

In this study, it is found that the N3(E)L mutation in the N-terminal linker of cpEGFP in NTS sensors greatly promotes ESPT occurrence in the sensor, and similar results are obtained on SST sensors. The question arises: can this pattern be generalized to other neuropeptide sensors, enabling large-scale development of ratiometric neuropeptide sensors?Based on this hypothesis, the PACAP1.0 sensor is selected as a mutation template. First, structural predictions are performed for the cpEGFP and its flanking linker sequences of both the wild-type PACAP1.0 sensor and the PACAP1.0-N3(E)L mutant. The results reveal that the N3(E)L mutation can also form two hydrogen bonds with the side chain of the S205 residue, and hydrogen bonding between S205 and E222 can be observed. These findings suggest that the N3(E)L mutation is likely to enhance ESPT in the PACAP1.0 sensor. Therefore, saturation mutagenesis screening is designed at the N3(E) position of PACAP1.0. The results show that when glutamic acid (E) at N3(E) is mutated to isoleucine (I), leucine (L), or methionine (M), all three mutants exhibit significant improvements in ΔF/F₀ (488), ΔF/F₀ (405), and ΔR/R₀ (FIG. 14C). To further determine which mutation yields the best performance, concentration-dependent response curves are measured for these three mutants. Results show that when glutamic acid (E) is mutated to leucine (L), the mutant had the greatest improvement in response amplitude under both Ex405 and Ex488, being −35% and 1500% respectively, with a ratio response close to 3000% (FIG. 14D). In summary, the N3(E)L mutation enhances the response of the PACAP sensor in a manner highly similar to its effect on the NTS sensor. In this study, the amino acid sequence of cpEGFP connected to the GPCR specifically binding PACAP is shown as SEQ ID NO: 3: NVYIKADKQKN GIKANFHIRINIEDGGVQLAYHYQQNTPIGDGPVLLPDNHYLSVQSKLSKDPNEKRDH MVLLEFVTAGTTLGMDELYKGGTGGSMVRKGEELFTGVVPILVELDGDVNGHKFSV SGEGEGDATYGKLTLKFICTIGKLPVPWPTLVTTLTYGVQCFSRYPDHNKQHDFFKSA MPEGYVQERTIFFKDDGNYKTRAEVKFEGDTLVNRELEKGIDFKEDGNILGHKLEYN; its N-terminal linker is LEEGG, C-terminal linker is TGAAA; the amino acid sequence of the PACAP1.0 sensor is shown as SEQ ID NO: 9: METDTLLLWULLWWPGSTGDTSLYKKV GTTGMGVVHVSLAALLLLPMAPAMISDCIFKKEQAMCLEKTQRANELMGFND SSPG CPGMVDNITCWRPAHVGEMVLVSCPELFRIFNPQQWETETIGESDFGDSNSLDLSDM GVVSNKCTEDGWSEPPPHYFDACGFDEYESETGDQDYYYLSVKALYYVGYSTSLVT LTTAMVILCRFRKLICTRNFHIMNLEVSPMLRATSVFIKDIVILYAEQDSNHCFISTVEC KAVMVFFHYCVVSNYFWLFLEGLYLFTLLVETSFPPERRVFVWYTIIGWGTPTVCVT VWATLRLYPDTGCMDMNDSTALWWYKGPVVGSIMVNPVLFIGIIRTILLQKLTSPAP AGPRQTDALDLEEGGWYIKADKQKNGIKANFHIRINIEDGGVQLAYHYQQNTPIGDG PVLLPDNHYLSVQSKLSKDPNEKRDHMVLLEFVTAGTTLGMDELYKGGTGGSMVR KGEELFTGVVPILVELDGDVNGHKFSVSGEGEGDATYGKLTLKFICTIGKLPVPWPTL VTTLTYGVQCFSRYPDHNKQHDFFKSAMPEGYVQERTIFFKDDGNYKTRAEVKFEG DTLVNRELEKGIDFKEDGNILGHKLEYNTGAAARWRASQYKRLARSTLLLIPLFGIHY IYTFAPSPENVSKRERLVFELGLOSFQGFVVAVLYCFLNGEVQAEIKRKWRSWKVNR YFAVDFKHRHPSLASSGVNGGTQLSILSKSSSQIRMSGLPADNLAT.

Additionally, observing new-generation NPY neuropeptide sensors reveal the same pattern. Structural predictions of cpEGFP of NPY1.0 and NPY1.3 sensors and their peptide-linker sequences show that in the NPY1.3 sensor, N3(E)L can form hydrogen bonds with the S205 side chain (FIG. 15A), and its response amplitude under Ex405 is significantly improved relative to NPY1.0, about −30% (FIG. 15B). In this study, the amino acid sequence of cpEGFP connected to the GPCR specifically binding NPY is shown as SEQ ID NO: 4: NVYIKADKQKNGIKANFHIRINIEDGGVQLAYHYQQNTPIGDGPVLLPDNHYLSVQS KLSKDPNEKRDHMVLLEFVTAGTTLGMDELYKGGTGGSMVRKGEELFTGVVPILVE LDGDVNGHKFSVSGEGEGDATYGKLTLKFICTIGKLPVPWPTLVTTLTYGVQCFSRY PDHNKQHDFFKSAMPEGYVQERTIFFKDDGNYKTRAEVKFEGDTLVNRELEKGIDFK EDGNILGHKLEYN; its N-terminal linker is LEEGG, C-terminal linker is TGAAA; the amino acid sequence of the NPY1.0 sensor before N3(E)L mutation is METDTLLLWULLW WPGSTGDTSLYKKVGTTGOSTLFSQVENHSVHSNFSEKNAQLLAPENDDCHLPLAMI FTLALAYGAVIILGVSSNLALIIIILKQKEMRNVTVNILIVNLSFSDLLVATTCLPFTFVT YLMDHWVFGEAMCKLNPPVQCVSITVSTFSLVLIAVERHQLIINPRGWRPNNRHAYV GIAVIIWLAVASSLPFLIYQVMTDEPFQNVTLDAYKDKYVCFDQFPSDSHRLSYTTLL LVLQVFGPLCFIFICYFKIVIRLKRRNMMPPSRRGPDAVAAPPGGTERRPNGLGPERSA GPGGAEAEPLPTQLNGAPGEPAPAGPRQTDALDLEEGGWYIKADKQKNGIKANFHIR INTEDGGVQLAYHYQQNTPIGDGPVLLPDNHYLSVQSKLSKDPNEKRDHMVLLEFVT AGTTLGMDELYKGGTGGSMVRKGEELFTGVVPILVELDGDVNGHKFSVSGEGEGDA TYGKLTLKFICTIGKLPVPWPTLVTTLTYGVQCFSRYPDHNKQHDFFKSAMPEGYVQ ERTIFFKDDGNYKTRAEVKFEGDTLVNRELEKGIDFKEDGNILGHKLEYNTGAAARW RGRQNETKRINIMLSTVVAFAVCWLPLTIFNYVFDWNHQIIATCNHNLIFLCHLTAM MSTCVNPIFYGFLNKNFQRDLQFFFNFCDFRSRDDDYETIAMSTMHTDVSKTSLKQA SPVAFKKINNDDNEKI (SEQ ID NO: 10).

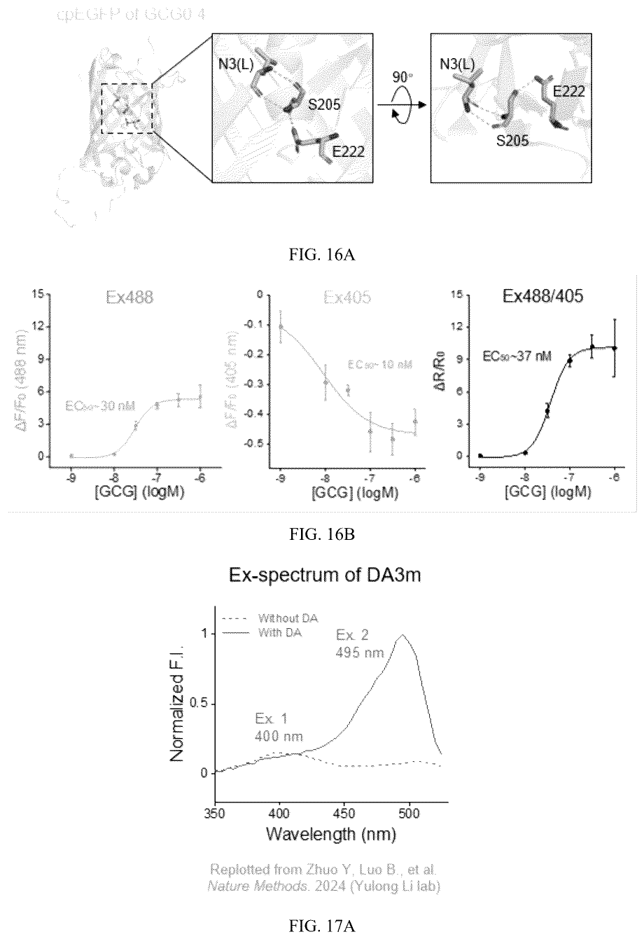

Similarly, the third amino acid of the N-terminal linker of cpEGFP in the GCG0.4 sensor corresponds to leucine (L). This sequence might confer excitation ratiometric properties to the GCG sensor, enabling ESPT possibility. Based on this hypothesis, structural prediction of cpEGFP in the GCG0.4 sensor and its peptide-linker sequences is performed. It is found that N3(L) can form two hydrogen bonds with the S205 side chain, and a hydrogen bond between S205 and E222 is observed (FIG. 16A). Further detection of concentration-dependent curves of the GCG0.4 sensor show that as externally added GCG concentration increases, its response amplitude under Ex488 excitation gradually increases, while under Ex405 excitation it gradually decreases. The maximum response amplitudes of the GCG0.4 sensor under Ex488 and Ex405 excitation are 600% and −45% respectively, with a maximum ratio response of about 1000% (FIG. 16B).

In summary, in this study, it is found that when the third amino acid (N3) of the N-terminal linker of cpEGFP in the GRAB neuropeptide sensor is leucine (L), it can significantly increase ΔF/F₀ (405) and ΔR/R₀ of the sensor. This pattern has been validated in multiple GRAB neuropeptide sensors (including NTS, PACAP, SST, NPY, and GCG sensors).

### Embodiment 4: Extending the Ratiometric Fluorescent Sensor Strategy to Monoamine Neurotransmitter Sensors

To systematically enhance the ESPT of GRAB sensors, this study further extends the ratiometric sensor development strategy to monoamine sensors beyond neuropeptide sensors.

Dopamine is an important monoamine neurotransmitter widely present in the central nervous system (CNS) and peripheral nervous system. Dopamine synthesis starts from the tyrosine, which is hydroxylated by tyrosine hydroxylase to form levodopa (L-DOPA, also referred to as L-3,4-dihydroxyphenylalanine), then decarboxylated by aromatic L-amino acid decarboxylase to form dopamine. Dopamine metabolism primarily occurs via monoamine oxidase (MAO) and catechol-O-methyltransferase (COMT), producing metabolites like 3,4-dihydroxyphenylacetic acid (DOPAC) and homovanillic acid (HVA).

As a neurotransmitter, dopamine transmits signals between neurons, influencing various physiological and psychological functions. Dopaminergic neurons are mainly concentrated in several central regions, including the substantia nigra pars compacta (SNc), ventral tegmental area (VTA), and arcuate nucleus. These dopaminergic neurons project to multiple brain regions via different pathways. (1) Nigrostriatal pathway: the dopaminergic neurons project from substantia nigra to striatum, primarily involved in motor control. Degeneration of dopaminergic neurons in this pathway is the main cause of Parkinson's disease. (2) Mesolimbic pathway: the dopaminergic neurons project from VTA to nucleus accumbens, amygdala, and hippocampus, associated with reward mechanisms, pleasure, and addiction. From VTA to nucleus accumbens, amygdala, and hippocampus, this pathway plays an important role in drug addiction (e.g., cocaine and amphetamines). (3) Mesocortical pathway: the dopaminergic neurons project from VTA to prefrontal cortex, involved in cognitive function, decision-making, and emotional regulation, closely related to schizophrenia and depression. (4) Tuberoinfundibular pathway: the dopaminergic neurons project from arcuate nucleus to anterior pituitary, regulating hormone secretion of the hypothalamic-pituitary axis. Dopamine in this pathway inhibits prolactin secretion; its dysfunction may lead to hyperprolactinemia.

Therefore, dopamine's functions in the central nervous system are complex and diverse, involving motor control, emotional regulation, reward mechanisms, cognitive function, and endocrine regulation. Its distribution and function in different neural pathways make it key for research on various neuropsychiatric disorders and drug addiction. Understanding these mechanisms of dopamine is crucial for developing effective treatments, thus detection of dopamine also requires more sensitive tools with high spatiotemporal resolution.

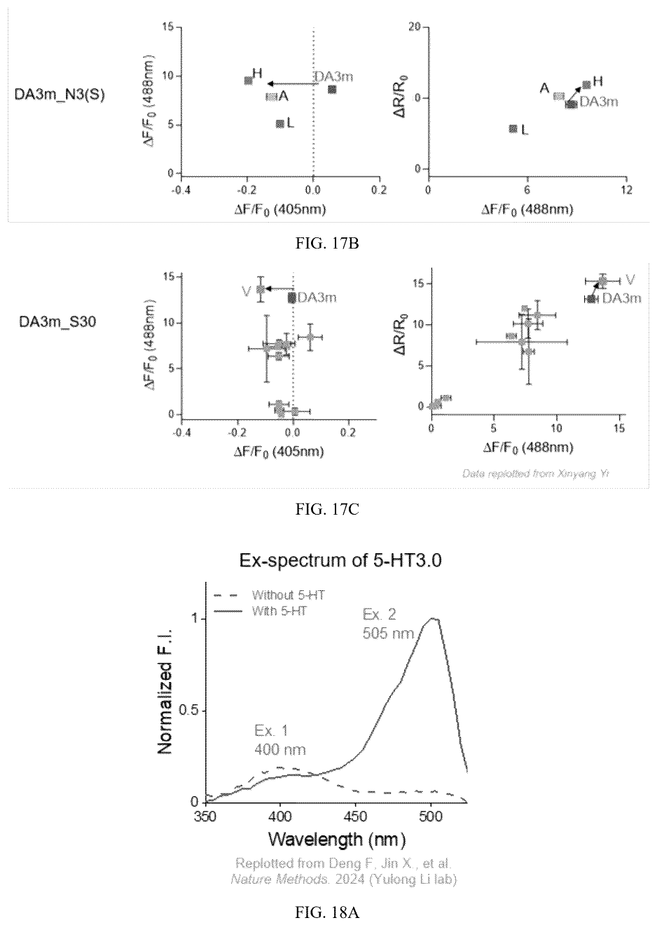

This study is based on the latest generation of green dopamine sensors, DA3m. Its excitation spectrum shows that DA3m exhibits negligible response to dopamine under 400 nm excitation (FIG. 17A), with a response amplitude nearly zero. Therefore, mutagenesis experiments targeting the N3(S) and S30 positions (corresponding to position 127 in cpEGFP) of DA3m are designed, aiming to develop a dopamine sensor with improved excitation ratiometric properties. Screening results show that when the N3(S) residue of DA3m is mutated to leucine (L), alanine (A), or histidine (H), the ΔF/F₀ (405) values of these three mutant sensors are significantly enhanced compared to DA3m, with the N3(S)L mutation providing the greatest improvement for ΔF/F₀ (405) and ΔR/R₀ (FIG. 17B). Additionally, screening at the S30 position reveals that the S30V mutant sensor exhibits significantly higher ΔF/F₀(405) and ΔR/R₀ than DA3m (FIG. 17C). In the disclosure, the amino acid sequence of cpEGFP connected to the GPCR specifically binding dopamine is shown as SEQ ID NO: 5: YVYIKADKQKNGIKANFGTKINIEDGGVQLAYHYQQNTPIGDGPVLLPDNHYLSVQS KLSKDPNEKRDHMVLLEFVTAGTTLGMDELYKGGTGGLSMVRKGEELFTGVVPILV ELDGDVNGHKFSVSGEGEGDATYGKTLYLKFICTIGKLPVPWPTLVTTLTYGVQCFS RYPDHNKQHDFFKSAMPEGYVQERTIFFKDDGNYKTRAEVKFEGDTLVNRTELEGT DFKEDGNILGHKLEYN; its N-terminal linker is LNSLL, C-terminal linker is NHDQL (SEQ ID NO: 24); the amino acid sequence of the dopamine sensor DA3m is METDTLLWVLLWPGSTGDTSLYKKVGTGEPLPPASTPSWNASSPGAASGGDNRTLV GPASAGARAVLPVLYLLCAAGLGNTLVIYVLRFAKMTVTNIYLNLAVADLYMLGLP FLATQNAASFWPPGPVLCRLVMLDGVNQFTSVCLTMSVDRYLAVVHPLSSARWRPR VAKLASAAWVLSLCMSLPLLFADVQEGGTCNASWPEVPLGAVFIITYAVLGFAPLLV ICLCYLIVKVRAAGVRGCALDELGGNYVYIKADQKNGIKANFGIRHNIEDGVQLAYH YQQNTPIDGPVLLPDNHYLSVQSKLSKDPNEKRDMVLFEFTAAIGITLGMDLYKGGT GGLMVSKGEELFTGVPILVELGDVNHKFSVSGEGEDATYGKLTLKFICTTGKLPVWP TLVTTLTYGVQCSFRYPDHMKQHDFKSAMPEGYIQERTIFFKNDGFYKTRAEVKFEG DTLVNRIELKGIDFKEDGNILGHKLEYNHDQLKRETKVKLTSLVIMGFVCCWLPFFIL NCILPFCGSGETQPCIDSNTFDVFVFWGANSSLPNIIYAFNADFRKAFSTLLGCYRLCP ATNNATETVSSINNNGAAMFSSHEPRGSISKECNLVLIPHAVGSSEDLKKEEAAGIARPL E KLSPALSVILYDTDVSLKIQPITQNGQHPT (SEQ ID NO: 11).

Serotonin (5-HT) is another important monoamine neurotransmitter widely present in the central nervous system (CNS) and peripheral nervous system. Serotonin is synthesized from tryptophan via tryptophan hydroxylase to 5-hydroxytryptophan (5-HTP), then decarboxylated by aromatic L-amino acid decarboxylase. Serotonin metabolism primarily occurs via monoamine oxidase (MAO), producing 5-hydroxyindoleacetic acid (5-HIAA).

Serotonin plays key roles in regulating mood, appetite, sleep, memory, and learning. Serotonergic neurons are primarily located in several nuclei of the midbrain and brainstem, especially the raphe nuclei. These neurons are distributed in raphe nucleus clusters, particularly the dorsal raphe and magnus raphe, projecting to various brain regions including cortex, limbic system, and spinal cord. Serotonin functions include the following aspect. (1) Mood regulation: serotonin plays a key role in regulating mood and emotion. Clinically, low serotonin levels are associated with mood disorders like depression and anxiety. Selective serotonin reuptake inhibitors (SSRIs) are common antidepressants that alleviate symptoms by increasing serotonin levels in the synaptic cleft. (2) Sleep regulation: serotonin involves in regulating sleep-wake cycles, especially serotonin's precursor (e.g., melatonin) plays an important role in promoting sleep. In addition, serotonin dysfunction can lead to sleep disorders like insomnia or irregular sleep. (3) Appetite control: serotonin plays an important role in regulating appetite and satiety. Abnormal serotonin levels may lead to eating disorders like anorexia or bulimia. (4) Pain regulation: serotonin involves in pain signal modulation in both central and peripheral nervous systems. Serotonergic drugs can be used to alleviate chronic pain and migraines. (5) Cognition and memory: serotonin activity in prefrontal cortex and hippocampus is related to cognitive function, learning, and memory formation. Serotonin dysfunction is associated with cognitive impairment and memory decline. (6) Serotonin's role in drug addiction is also very important. Many addictive drugs, such as MDMA (ecstasy) and hallucinogens, produce effects by affecting the serotonin system. MDMA causes massive serotonin release and inhibits its reuptake, significantly increasing synaptic serotonin levels. This leads to intense pleasure and emotional empathy, but long-term use can deplete serotonin reserves, causing low mood and cognitive impairment. Hallucinogens activate serotonin receptors (e.g., 5-HT2A receptor), causing profound changes in perception, emotion, and cognition, sometimes leading to psychosis-like symptoms.

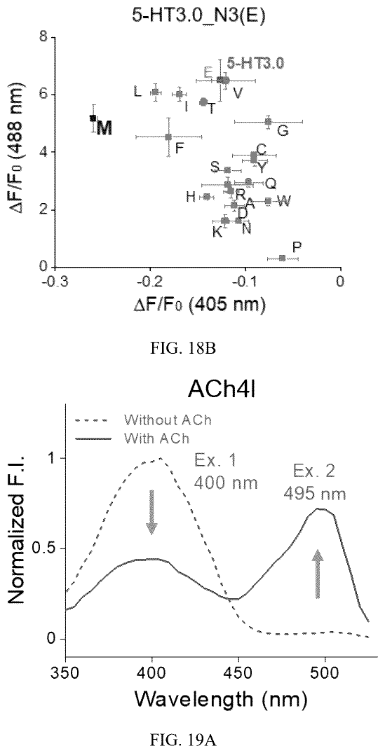

To develop sensitive excitation ratiometric 5-HT sensors, this study is based on the latest generation of green serotonin sensors, 5-HT3.0. Its excitation spectrum shows that 5-HT3.0 exhibits minimal response to 5-HT under 400 nm excitation (FIG. 18A). Therefore, mutagenesis experiments are designed targeting the N3(E) position of 5-HT3.0. Results show that when the N3(E) residue of 5-HT3.0 is mutated to methionine (M) or leucine (L), both mutant sensors exhibit significantly enhanced ΔF/F₀ (405) values compared to DA3m, with the N3(E)M mutation providing the greatest improvement for ΔF/F₀ (405) (FIG. 18B). In the disclosure, the amino acid sequence of cpEGFP connected to the GPCR specifically binding serotonin is shown as SEQ ID NO: 6: NYTKADKQKNGTKATFHTRINTEDGGVQLAYHY QQNTPTGDPPVLLPDNHYLSVQSKLSKDPNEKRDHMVLLEFVTAGTTLGMDELYKG GTGGSMVRKGEELFTGVVPILVELDGDVNGHKFSVRGEGEGDATNGKLTLYRTCTT GKLPVPWPTLVTTLTYGVQCFSRYPDHNKQHDFFKSAMPEGYVQERTIFFKDDGNY KTRAEVKFEGDTLVNRTELKGTDFKEDGNTLGHKLEYN; its N-terminal linker is LEEGG, C-terminal linker is TGAAA; the amino acid sequence of the serotonin sensor 5-HT3.0 is shown as SEQ ID NO: 12: METDTLLLWULLWWPGSTGDTSLYKKVGTTGD RLDANVSSNEGFRSVEWVLLTFLAVVLMATLGNLLVWVACRRQRLKKIKTNYFLVS LAFADLLVSVLVMPFGATELVQDIVAVGEMFCLKTSLDVCLCTASTFHLCCISLDRY YAATCQPLVYRNKMTPLRTALMLCGCWVLPWFISFLPLMQGNNNIGTVDVIERKRS HNSNTVCVFWVNFPYATTCSVVAFVTPFLLMVLAYTRTYVVAKRGPDAVAAPPGGT ERRPNGLGPERSAGPGGAEAEPLPTQLNGAPGFAPAGPRQTDALDLEEGGWYTKAD KQKNGTKATFHTRINTEDGGVQLAYHYQQNTPTGDPPVLLPDNHYLSVQSKLSKDP NEKRDHMVLLEFVTAGTTLGMDELYKGGTGGSMVRKGEELFTGVVPILVELDGDVN GHKFSVRGEGEGDATNGKLTLYRTCTTGKLPVPWPTLVTTLTYGVQCFSRYPDHNK QHDFFKSAMPEGYVQERTIFFKDDGNYKTRAEVKFEGDTLVNRTELKGTDFKEDGN TLGHKLEYNTGAAARFRWRTETKAAKVLCVIMGCFCFCWAPFFVTLVVDPFIDTYVP EQVWTAPLGVINSGLNPFLYAPLNKSPRRAFLITLRCNYKVEKKPPVRQIPRVAATAL SGRELNVNTYRHTNEPVTEKASDNEPGIEMQVENLELPVNPSSVWSERISSV.

Acetylcholine (ACh) is widely present in the central and peripheral nervous systems. Acetylcholine is synthesized from choline and acetyl-CoA by choline acetyltransferase (ChAT). Its degradation is primarily catalyzed by acetylcholinesterase (AChE), producing choline and acetate. Acetylcholine transmits signals between neurons, affecting muscle contraction, memory, attention, and autonomic nervous function. Cholinergic neurons in the CNS are mainly distributed in the basal forebrain (e.g., medial septal nucleus and nucleus basalis of Meynert) and brainstem (e.g., locus coeruleus of pons and hypothalamus); in the peripheral nervous system, mainly in the autonomic nervous system (including sympathetic and parasympathetic nerves) and motor nerve endings. Acetylcholine functions include the following aspect. (1) Muscle contraction: acetylcholine releases at neuromuscular junctions, activating muscle fiber contraction. Dysfunction of acetylcholine receptors leads to myasthenia gravis. (2) Memory and learning: acetylcholine plays a key role in hippocampus and cerebral cortex, promoting memory and learning. Clinically, Alzheimer's disease patients show significant decrease in acetylcholine levels. (3) Attention and arousal: acetylcholine regulates attention, arousal state, and cognitive function. Attention deficit hyperactivity disorder (ADHD) is related to acetylcholine dysfunction. (4) Autonomic function: acetylcholine regulates heart rate, digestion, and urinary function in the autonomic nervous system. Clinical association: autonomic dysfunctions like hypotension, arrhythmia are related to acetylcholine imbalance. Therefore, drugs regulating acetylcholine levels can treat corresponding diseases. For example, AChE inhibitors increase synaptic acetylcholine by inhibiting AChE activity, used to treat Alzheimer's and myasthenia gravis. Neurotoxins (e.g., botulinum toxin) block acetylcholine release, causing muscle relaxation, used to treat muscle spasms and cosmetic procedures.

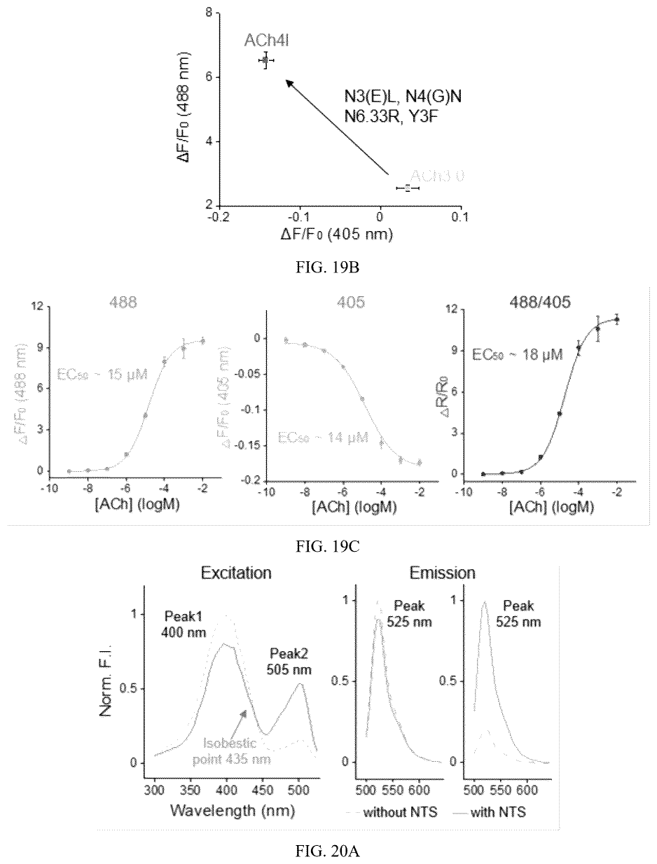

To better discover and understand acetylcholine function and action, sensitive excitation ratiometric ACh sensors also need development. This study found that the latest generation green acetylcholine sensor ACh41 is a good excitation ratiometric sensor, whose excitation spectrum shows ACh41 has a relatively large response amplitude to ACh under 400 nm excitation (FIG. 19A). To understand why ACh41 has ratiometric characteristics, the optimization process of the ACh41 sensor is reviewed, finding that ACh41 introduced four variants based on the previous ACh3.0 version: N3(E)L, N4(G)N, N6.33R, and Y3F (FIG. 19B). The concentration-dependent curves of the ACh41 sensor show that as ACh concentration increases, ΔF/F₀ (488) gradually increases, ΔF/F₀ (405) gradually decreases, and its ratio ΔR/R₀ also gradually increases. Moreover, the ratio ΔR/R₀ of the sensor response to 10 mM ACh is close to 1200%, significantly higher than its ΔF/F₀ (488) of 900% (FIG. 19C).

### Embodiment 5: Performance Characterization of NTS1.5 Sensor in HEK293T Cells

Ratiometric neuropeptide sensors exhibit opposite-direction responses upon binding neuropeptides when excited at 488 nm and 405 nm. Therefore, calculating the Ex488/Ex405 ratio can significantly enhance the response amplitude of the sensor, providing the potential for sensitive detection of dynamic neuropeptide changes. However, the advantage of ratiometric neuropeptide sensors is not only in their higher response amplitude but also in their ability to minimize fluorescence fluctuations caused by certain environmental factors (e.g., environmental pH, hemodynamics, movements, laser power). Since the sensor itself has only one fluorescent protein containing one chromophore, when external factors (pH, laser power, etc.) change, the sensor's fluorescence changes accordingly. However, because the fluorescence of a ratiometric sensor under 488 nm and 405 nm excitation comes from the same chromophore, the effect of external factors on Ex488 and Ex405 is in the same direction. Therefore, using the Ex488/405 ratio may help correct changes in fluorescence intensity caused by pH or laser power changes, thereby improving sensor stability and ensuring signal detection specificity.

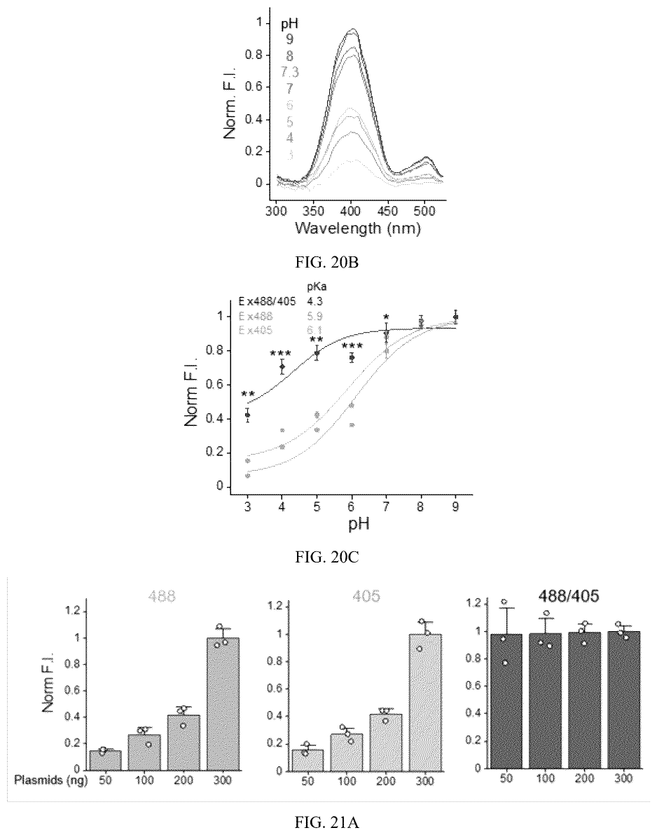

First, the spectral properties of the NTS1.5 sensor are characterized. Results show the NTS1.5 sensor has two excitation peaks at 400 nm and 505 nm, with corresponding emission peaks both at 525 nm (FIG. 20A). To verify the pH sensitivity of the NTS1.5 sensor, the pH of the cellular environment and intracellular pH are altered. Results show that as pH decreases, the sensor's fluorescence brightness under Ex488 and Ex405 also gradually decreases (FIG. 20B). In contrast, the Ex488/405 ratio is less affected by pH changes. For example, at pH=5, Ex488 is only 30% of maximum brightness, but Ex488/405 is 80% of maximum brightness, its fluorescence brightness is significantly higher than Ex488 (FIG. 20C). These results suggest the NTS1.5 sensor may be less affected by pH changes when detecting dynamic changes of neuropeptide release in vivo.

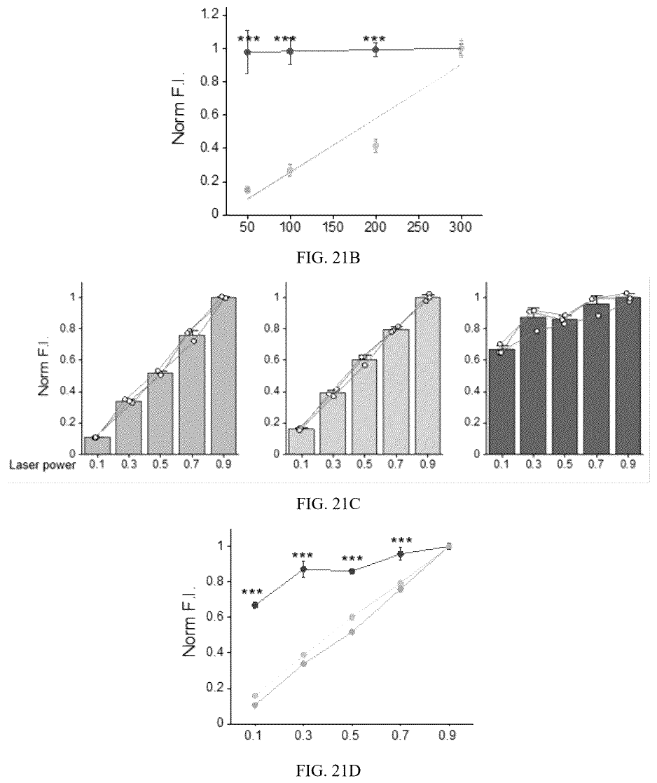

Additionally, fluorescent protein brightness is greatly affected by sensor expression level and laser power. For example, higher sensor expression leads to higher Ex488 and Ex405 of cpEGFP; higher laser power used in experiments leads to higher Ex488 and Ex405 of cpEGFP. Since the effects of sensor expression level and laser power on Ex488 and Ex405 of cpEGFP are in the same direction, it is hypothesized that Ex488/405 might remain stable. To test whether the fluorescence brightness of the NTS1.5 sensor is affected by sensor expression level, 50 nanograms (ng), 100 ng, 200 ng, and 300 ng of the NTS1.5 sensor are expressed in HEK293T cells, and Ex488, Ex405, and Ex488/405 are detected under different expression levels. Experimental results show that as sensor expression level increases, fluorescence brightness under Ex488 and Ex405 also gradually increases, but the Ex488/405 fluorescence brightness ratio remains stable (FIG. 21A-FIG. 21).

To further verify whether the fluorescence brightness of the NTS1.5 sensor is affected by laser power, 300 ng of NTS1.5 sensor is expressed in HEK293T cells, and laser powers of 10%, 30%, 50%, 70%, and 90% are used to excite the sensor, detecting Ex488, Ex405, and Ex488/405 under different laser power intensities. Experimental results show that as laser power increases, fluorescence brightness under Ex488 and Ex405 increases proportionally, but the Ex488/405 brightness ratio remains relatively stable. For example, at 10% laser power, Ex488 is 10% of maximum brightness, but Ex488/405 is 70% of maximum brightness, its fluorescence brightness is significantly higher than Ex488 (FIG. 21C-D).

### Embodiment 6: Performance Characterization of SST2.2 Sensor in HEK293T Cells

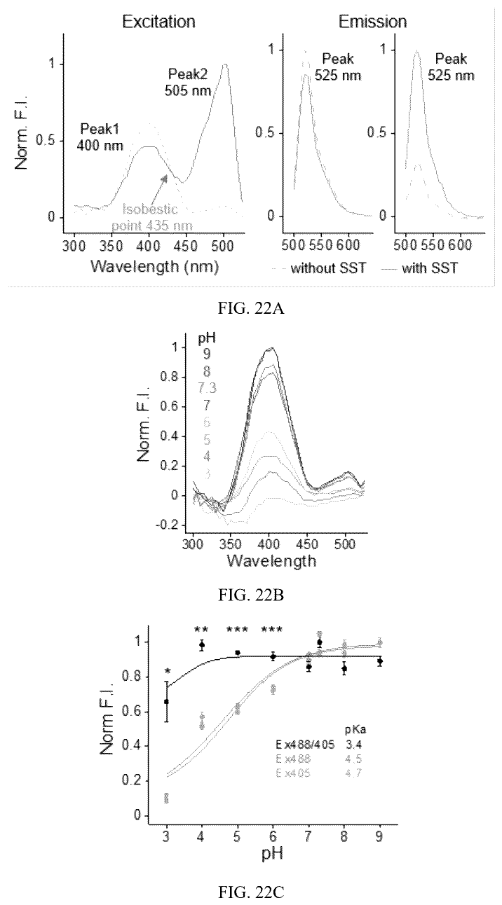

Similarly, the spectral properties of the SST2.2 sensor are characterized. SST2.2 sensor has two excitation peaks at 400 nm and 505 nm, with corresponding emission peaks both at 525 nm (FIG. 22A). To verify the pH sensitivity of the SST2.2 sensor, the pH of the cellular environment and intracellular pH are altered. Results show that as pH decreases, the sensor's fluorescence brightness under 488 and 405 nm also gradually decreases (FIG. 22B). In contrast, the Ex488/405 ratio is less affected by pH changes. Even at pH=3, Ex488 is only 10% of maximum brightness, but Ex488/405 remains 60% of maximum brightness, its fluorescence brightness is significantly higher than Ex488 (FIG. 22C). These results indicate the SST2.2 sensor has good pH sensitivity.

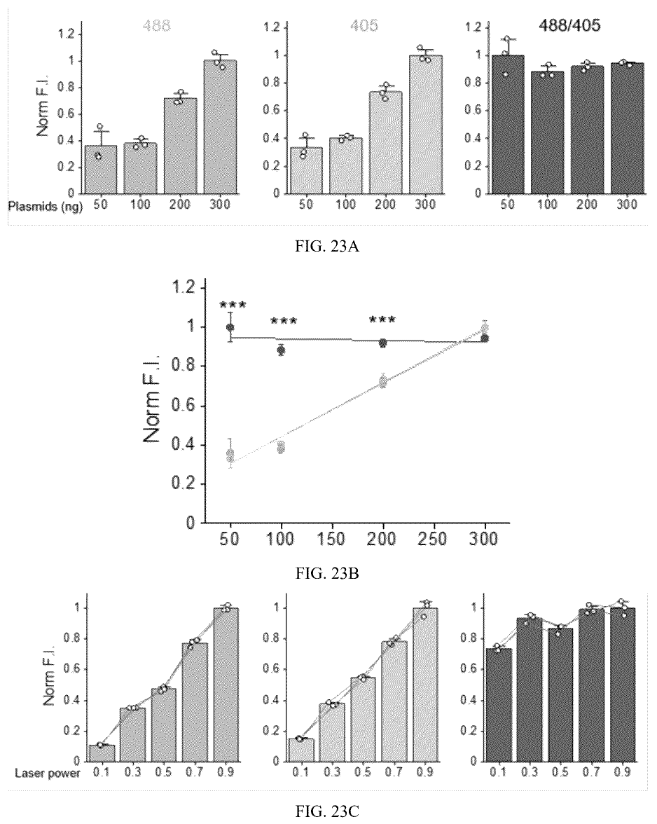

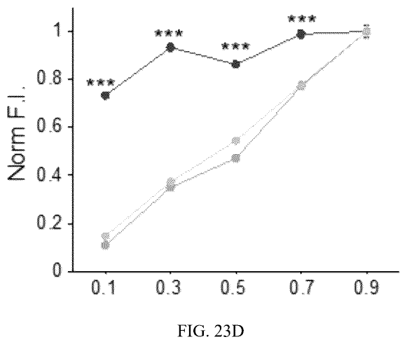

To test whether SST2.2 sensor fluorescence brightness is affected by sensor expression level, 50 ng, 100 ng, 200 ng, and 300 ng of SST2.2 sensor are expressed in HEK293T cells, and Ex488, Ex405, and Ex488/405 are detected under different expression levels. Experimental results show that as sensor expression level increases, Ex488 and Ex405 also gradually increase, but Ex488/405 remains stable (FIG. 23A-FIG. 23B). To further verify whether the fluorescence brightness of the SST2.2 sensor is affected by laser power, 300 ng of SST2.2 sensor is expressed in HEK293T cells, and laser powers of 10%, 30%, 50%, 70%, and 90% are used to excite the sensor, detecting Ex488, Ex405, and Ex488/405 under different laser power intensities. Experimental results show that as laser power increases, Ex488 and Ex405 increase proportionally, but Ex488/405 remains relatively stable. For example, at 10% laser power, Ex488 is 10% of maximum brightness, but Ex488/405 is 75% of maximum brightness, its fluorescence brightness is significantly higher than Ex488 (FIG. 23C-D).

The above provides exemplary description of the disclosure. It should be noted that any simple modifications, alterations, or other equivalent substitutions that can be made by those skilled in the art without creative effort fall within the protection scope of the disclosure, provided they do not depart from the core of the disclosure.

1. A ratiometric fluorescent sensor constructed based on a G protein-coupled receptor (GPCR), wherein the ratiometric fluorescent sensor comprises the GPCR, a circularly permuted fluorescent protein, and peptide-linkers; the peptide-linkers comprise an N-terminal linker and a C-terminal linker; the circularly permuted fluorescent protein is inserted between a fifth transmembrane domain and a sixth transmembrane domain of the GPCR, an N-terminus of the circularly permuted fluorescent protein is connected to the fifth transmembrane domain of the GPCR via the N-terminal linker; and a C-terminus of the circularly permuted fluorescent protein is connected to the sixth transmembrane domain of the GPCR via the C-terminal linker;
the N-terminal linker comprises 5 amino acids, and a third amino acid of the N-terminal linker is mutated;
the ratiometric fluorescent sensor is capable of being expressed on a cell membrane; and the ratiometric fluorescent sensor is capable of binding to a specific ligand of the GPCR when in contact with the specific ligand of the GPCR, thereby causing a detectable change in a fluorescence intensity of the ratiometric fluorescent sensor;
the GPCR is of human origin;
the specific ligand is a neurotransmitter;
the neurotransmitter comprises at least one selected from the group consisting of neurotensin (NTS), somatostatin (SST), pituitary adenylate cyclase-activating polypeptide (PACAP), neuropeptide Y (NPY), dopamine, and serotonin;
the circularly permuted fluorescent protein is a circularly permuted enhanced green fluorescent protein (cpEGFP);
when the cpEGFP is connected to the GPCR specifically binding the NTS to obtain a first resulting protein, the first resulting protein has the amino acid sequence as shown in SEQ ID NO: 1 or a variant thereof with a glutamic acid substitution at position 127;
when the cpEGFP is connected to the GPCR specifically binding the SST to obtain a second resulting protein, the second resulting protein has the amino acid sequence as shown in SEQ ID NO: 2 or a variant thereof with a glutamic acid substitution at position 127;
when the cpEGFP is connected to the GPCR specifically binding the PACAP to obtain a third resulting protein, the third resulting protein has the amino acid sequence as shown in SEQ ID NO: 3;
when the cpEGFP is connected to the GPCR specifically binding the NPY to obtain a fourth resulting protein, the fourth resulting protein has the amino acid sequence as shown in SEQ ID NO: 4;
when the cpEGFP is connected to the GPCR specifically binding the dopamine to obtain a fifth resulting protein, the fifth resulting protein has the amino acid sequence as shown in SEQ ID NO: 5 or a variant thereof with a valine substitution at position 127;
when the cpEGFP is connected to the GPCR specifically binding the serotonin to obtain a sixth resulting protein, the sixth resulting protein has the amino acid sequence as shown in SEQ ID NO: 6;
when the cpEGFP is connected to the GPCR specifically binding the NTS, the SST, the PACAP, the NPY, or the serotonin, the sequence of the N-terminal linker before mutation of the third amino acid is LEEGG;
when the cpEGFP is connected to the GPCR specifically binding the NTS, the SST, or the NPY, the third amino acid of the N-terminal linker is mutated to leucine, and the mutated sequence of the N-terminal linker is LELGG;
when the cpEGFP is connected to the GPCR specifically binding the PACAP, the third amino acid of the N-terminal linker is mutated to leucine, isoleucine, or methionine, and the mutated sequence of the N-terminal linker is any one of LELGG, LEIGG, or LEMGG;
when the cpEGFP is connected to the GPCR specifically binding the serotonin, the third amino acid of the N-terminal linker is mutated to leucine or methionine, and the mutated sequence of the N-terminal linker is any one of LELGG or LEMGG;
when the cpEGFP is connected to the GPCR specifically binding the dopamine, the sequence of the N-terminal linker before mutation of the third amino acid is LNSLI;
when the cpEGFP is connected to the GPCR specifically binding the dopamine, the third amino acid of the N-terminal linker is mutated to leucine, alanine, or histidine, and the mutated sequence of the N-terminal linker is any one of LNLLI, LNALI, or LNHLI.

2. A method for constructing a ratiometric GPCR-activation-based (GRAB) fluorescent sensor, wherein the method comprises mutating a third amino acid of a N-terminal linker of a circularly permuted fluorescent protein connected to a GPCR;
the circularly permuted fluorescent protein is cpEGFP;
the mutating comprises mutating the third amino acid of the N-terminal linker of the cpEGFP to leucine;
when the cpEGFP is connected to the GPCR specifically binding NTS, SST, PACAP, NPY, or serotonin, the sequence of the N-terminal linker before mutation of the third amino acid is LEEGG;
when the cpEGFP is connected to the GPCR specifically binding dopamine, the sequence of the N-terminal linker before mutation of the third amino acid is LNSLI.

3. The method as claimed in claim 2, wherein when the GPCR is the GPCR specifically binding the PACAP, the mutating further comprises mutating the third amino acid of the N-terminal linker of the cpEGFP to isoleucine or methionine;
when the GPCR is the GPCR specifically binding the dopamine, the mutating further comprises mutating the third amino acid of the N-terminal linker of the cpEGFP to alanine or histidine;
when the GPCR is the GPCR specifically binding the serotonin, the mutating further comprises mutating the third amino acid of the N-terminal linker of the cpEGFP to methionine.

4. The method as claimed in claim 2, wherein when the GPCR is the GPCR specifically binding the NTS or the SST, the method further comprises mutating a 127^{th} site of the cpEGFP, the mutating comprising mutating the 127^{th} site of the cpEGFP to glutamic acid;
when the GPCR is a GPCR specifically binding the dopamine, the method further comprises mutating the 127^{th} site of the cpEGFP to valine.

5. A biological material, wherein the biological material comprises:
1) a polynucleotide, wherein the polynucleotide encodes the ratiometric fluorescent sensor as claimed in claim 1;
2) an expression vector, wherein the expression vector comprises the polynucleotide of 1);
3) a host cell, wherein the host cell comprises the polynucleotide of 1) or the expression vector of 2).

6. Any one of the following applications, wherein the applications comprise:
1) an application of the method as claimed in claim 2 in constructing the ratiometric GRAB fluorescent sensor;
2) an application of a ratiometric fluorescent sensor in detection of concentration changes of a specific ligand of GPCR in a sample to be tested or a tissue to be tested;
3) application of the ratiometric GRAB sensor constructed by the method in detection of the concentration changes of the specific ligand of the GPCR in the sample to be tested or the tissue to be tested;
the specific ligand comprises a neurotransmitter;
the neurotransmitter comprises the NTS, the SST, the PACAP, the NPY, the dopamine, and/or the serotonin;
the detection comprises qualitative and/or quantitative detection.


SEQUENCE LISTING (24 sequences)

SEQ ID NO: 1 - protein, 241 aa, synthetic construct
NVYIKADKQKNGIKAAFNIRHNIEDGGVQLAYHYQQNTPIGDGPVLLPDNHYLSVQSKLSKDPNEKRDHMVLLEFVTAAGITWGMDELYKGGTGGSMVRKGEELFTGVVPILVELDGDVNGHKFSVGGEGEGDATEGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 2 - protein, 241 aa, synthetic construct
NVYIKADKQKNGIKANFHIRHNIEDGGVQLAYHYQQNTPIGDGPVLLPDNHYLSVQSKLSKDPNEKRDHMVLLEFVTAAGITIGMDELYKGGTGGSMVRKGEELFTGVVPILVELDGDVNGHKFSVSGEGEGDATEGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 3 - protein, 241 aa, synthetic construct
NVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 4 - protein, 241 aa, synthetic construct
NVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATEGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYN

SEQ ID NO: 5 - protein, 241 aa, synthetic construct
YVYIKADKQKNGIKANFGIRHNIEDGGVQLAYHYQQNTPIGDGPVLLPDNHYLSVQSKLSKDPNEKRDHMVLLEFVTAAGITLGMDELYKGGTGGLMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKNDGFYKTRAEVKFEGDTLVNRIELKGIDFKEDGNILGHKLEYN

SEQ ID NO: 6 - protein, 241 aa, synthetic construct
NVYIKADKQKNGIKAIFHIRHNIEDGGVQLAIHYQQNTPIGDGPVLLPDNHYLSVQSKLSKDPNEKRDHMVLLEFVTAAGITLGMDELYKGGTGGSMVRKGEELFTGVVPILVELDGDVNGHKFSVRGEGEGDATNGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYN

SEQ ID NO: 7 - protein, 708 aa, synthetic construct
METDTLLLWVLLLWVPGSTGDTSLYKKVGTTGRLNSSAPGTPGTPAADPFQRAQAGLEEALLAPGFGNASGNASERVLAAPSSELDVNTDIYSKVLVTAVYLALFVVGTVGNTVTAFTLARKKSLQSLQSTVHYHLGSLALSDLLTLLLAMPVELYNFIWVHHPWAFGDAGCRGYYFLRDACTYATALNVASLSVERYLAICHPFKAKTLMSRSRTKKFISAIWLASALLAVPMLFTMGEQNRSADGQHAGGLVCTPTIHTATVKVVIQVNTFMSFIFPMVVISVLNTIIANKLTVMVRQAAEQLNGAPGEPAPAGPRDTDALDLEEGGNVYIKADKQKNGIKAAFNIRHNIEDGGVQLAYHYQQNTPIGDGPVLLPDNHYLSVQSKLSKDPNEKRDHMVLLEFVTAAGITWGMDELYKGGTGGSMVRKGEELFTGVVPILVELDGDVNGHKFSVGGEGEGDATEGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNTGATRRWRGREPNRVQALRHGVRVLRAVVIAFVVCWLPYHVRRLMFCYISDEQWTPFLYDFYHYFYMVTNALFYVSSTINPILYNLVSANFRHIFLATLACLCPVWRRRRKRPAFSRKADSVSSNHTLSSNATRETLY

SEQ ID NO: 8 - protein, 649 aa, synthetic construct
METDTLLLWVLLLWVPGSTGDTSLYKKVGTTGEPLFPASTPSWNASSPGAASGGGDNRTLVGPAPSAGARAVLVPVLYLLVCAAGLGGNTLVIYVVLRFAKMKTVTNIYILNLAVADVLYMLGLPFLATQNAASFWPFGPVLCRLVMTLDGVNQFTSVFCLTVMSVDRYLAVVHPLSSARWRRPRVAKLASAAAWVLSLCMSLPLLVFADVQEGGTCNASWPEPVGLWGAVFIIYTAVLGFFAPLLVICLCYLLIVVKVRAAGVRVGCALDLELGGNVYIKADKQKNGIKANFHIRHNIEDGGVQLAYHYQQNTPIGDGPVLLPDNHYLSVQSKLSKDPNEKRDHMVLLEFVTAAGITIGMDELYKGGTGGSMVRPGEELFTGVVPILVELDGDVNGHKFSVSGEGEGDATEGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNTGAAKRWRGRQLKVTWMVLVVVLVFAGCWLPFFTVNIVNLAVALPQEPASAGLYFFVVILSYANSCANPVLYGFLSDNFRQSFQKVLCLRKGSGAKDADATEPRPDRIRQQQEATPPAHRAAANGLMQTSKL

SEQ ID NO: 9 - protein, 761 aa, synthetic construct
METDTLLLWVLLLWVPGSTGDTSLYKKVGTTGMAGVVHVSLAALLLLPMAPAMHSDCIFKKEQAMCLEKIQRANELMGFNDSSPGCPGMWDNITCWKPAHVGEMVLVSCPELFRIFNPDQVWETETIGESDFGDSNSLDLSDMGVVSRNCTEDGWSEPFPHYFDACGFDEYESETGDQDYYYLSVKALYTVGYSTSLVTLTTAMVILCRFRKLHCTRNFIHMNLFVSFMLRAISVFIKDWILYAEQDSNHCFISTVECKAVMVFFHYCVVSNYFWLFIEGLYLFTLLVETFFPERRYFYWYTIIGWGTPTVCVTVWATLRLYFDDTGCWDMNDSTALWWVIKGPVVGSIMVNFVLFIGIIRILLQKLTSIPAPAGPRDTDALDLEEGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNTGAAARWRASQYKRLARSTLLLIPLFGIHYTVFAFSPENVSKRERLVFELGLGSFQGFVVAVLYCFLNGEVQAEIKRKWRSWKVNRYFAVDFKHRHPSLASSGVNGGTQLSILSKSSSQIRMSGLPADNLAT

SEQ ID NO: 10 - protein, 725 aa, synthetic construct
METDTLLLWVLLLWVPGSTGDTSLYKKVGTTGNSTLFSQVENHSVHSNFSEKNAQLLAFENDDCHLPLAMIFTLALAYGAVIILGVSGNLALIIIILKQKEMRNVTNILIVNLSFSDLLVAITCLPFTFVYTLMDHWVFGEAMCKLNPFVQCVSITVSIFSLVLIAVERHQLIINPRGWRPNNRHAYVGIAVIWVLAVASSLPFLIYQVMTDEPFQNVTLDAYKDKYVCFDQFPSDSHRLSYTTLLLVLQYFGPLCFIFICYFKIYIRLKRRNNMMPPSRRGPDAVAAPPGGTERRPNGLGPERSAGPGGAEAEPLPTQLNGAPGEPAPAGPRDTDALDLEEGGNVYIKADKQKNGIKANFHIRHNIEDGGVQLAYHYQQNTPIGDGPVLLPDNHYLSVQSKLSKDPNEKRDHMVLLEFVTAAGITLGMDELYKGGTGGSMVRKGEELFTGVVPILVELDGDVNGHKFSVSGEGEGDATEGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNTGAAARWRGRQNETKRINIMLLSIVVAFAVCWLPLTIFNTVFDWNHQIIATCNHNLLFLLCHLTAMMSTCVNPIFYGFLNKNFQRDLQFFFNFCDFRSRDDDYETIAMSTMHTDVSKTSLKQASPVAFKKINNNDDNEKI

SEQ ID NO: 11 - protein, 688 aa, synthetic construct
METDTLLLWVLLLWVPGSTGDTSLYKKVGTTGRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPARYERKMTPKAAFILISVAWTLSVLISFIPVQLSWHKAKPTSPSDGNATSLAETIDNCDSSLSRTYAISSSVISFYIPVAIMIVLYTRIYRIAQKQLNSLIYVYIKADKQKNGIKANFGIRHNIEDGGVQLAYHYQQNTPIGDGPVLLPDNHYLSVQSKLSKDPNEKRDHMVLLEFVTAAGITLGMDELYKGGTGGLMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKNDGFYKTRAEVKFEGDTLVNRIELKGIDFKEDGNILGHKLEYNNHDQLKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSLEKIQPITQNGQHPT

SEQ ID NO: 12 - protein, 716 aa, synthetic construct
METDTLLLWVLLLWVPGSTGDTSLYKKVGTTGDKLDANVSSNEGFRSVEKVVLLTFLAVVILMAILGNLLVMVAVCRDRQLRKIKTNYFIVSLAFADLLVSVLVMPFGAIELVQDIWAYGEMFCLVRTSLDVYLCTASIFHLCCISLDRYYAICCQPLVYRNKMTPLRIALMLGGCWVLPMFISFLPIMQGWNNIGIVDVIEKRKFSHNSNSTWCVFMVNKPYAITCSVVAFYIPFLLMVLAYYRIYVVAKRGPDAVAAPPGGTERRPNGLGPERSAGPGGAEAEPLPTQLNGAPGEPAPAGPRDTDALDLEEGGNVYIKADKQKNGIKAIFHIRHNIEDGGVQLAIHYQQNTPIGDGPVLLPDNHYLSVQSKLSKDPNEKRDHMVLLEFVTAAGITLGMDELYKGGTGGSMVRKGEELFTGVVPILVELDGDVNGHKFSVRGEGEGDATNGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNTGAAARFRWRTETKAAKVLCVIMGCFCFCWAPFFVTNIVDPFIDYTVPEQVWTAFLWLGYINSGLNPFLYAFLNKSFRRAFLIILRCNYKVEKKPPVRQIPRVAATALSGRELNVNIYRHTNEPVIEKASDNEPGIEMQVENLELPVNPSSVVSERISSV

SEQ ID NO: 13 - protein, 5 aa, synthetic construct
LEEGG

SEQ ID NO: 14 - protein, 5 aa, synthetic construct
LELGG

SEQ ID NO: 15 - protein, 5 aa, synthetic construct
LEIGG

SEQ ID NO: 16 - protein, 5 aa, synthetic construct
LEMGG

SEQ ID NO: 17 - protein, 5 aa, synthetic construct
LNSLL

SEQ ID NO: 18 - protein, 5 aa, synthetic construct
LNLLL

SEQ ID NO: 19 - protein, 5 aa, synthetic construct
LNALI

SEQ ID NO: 20 - protein, 5 aa, synthetic construct
LNALI

SEQ ID NO: 21 - protein, 5 aa, synthetic construct
ETEGG

SEQ ID NO: 22 - protein, 5 aa, synthetic construct
TGATR

SEQ ID NO: 23 - protein, 5 aa, synthetic construct
TGAAA

SEQ ID NO: 24 - protein, 5 aa, synthetic construct
NHDQL